(19) United States
(12) Patent Application Publication
(10) Pub. No.: US 2014/0275060 A1
(43) Pub. Date: Sep. 18, 2014

(54) COMPOUNDS FOR THE TREATMENT OF NEUROLOGIC DISORDERS

(71) Applicants: David Steven Menaldino, Atlanta, GA (US); George Walter Koszalka, Chapel Hill, NC (US); Ruppa Poornachary Kamalesh Babu, Alpharetta, GA (US); Scott James Myers, Avondale Estates (UA)

(72) Inventors: David Steven Menaldino, Atlanta, GA (US); George Walter Koszalka, Chapel Hill, NC (US); Ruppa Poornachary Kamalesh Babu, Alpharetta, GA (US); Scott James Myers, Avondale Estates (UA)

(21) Appl. No.: 14/210,771

(22) Filed: Mar. 14, 2014

Related U.S. Application Data
(60) Provisional application No. 61/784,419, filed on Mar. 14, 2013.

(51) Int. Cl. A61K 31/538; A61K 31/496; A61K 31/495; C07D 401/12; C07D 239/80; C07D 213/38; C07D 215/227; C07D 295/084; A61K 31/517; C07D 265/36

(52) U.S. Cl. CPC A61K 31/538; A61K 31/517; A61K 31/496; A61K 31/495; C07D 265/36; C07D 239/80; C07D 213/38; C07D 215/227; C07D 295/084; C07D 401/12
USPC 514/230.5; 514/252.17; 514/253.07; 514/253.11; 514/255.03; 544/105; 544/286; 544/360; 544/363; 544/393; 544/395

(57) ABSTRACT

Provided are compounds, pharmaceutical compositions and methods of treatment or prophylaxis of certain neurologic disorders, including disorders related to NMDA receptor activity, including neuropsychiatric disorders, neurodegenerative disorders and other neurologic diseases, disorders and conditions including stroke, brain injury, epilepsy, neuropsychiatric disorders, mood disorders, chronic pain and related conditions.

### FIELD OF THE ART

The present invention provides certain compounds useful in the treatment or prophylaxis of neurologic disorders, including neuropsychiatric disorders such as depression and anxiety, neurodegenerative disorders and other diseases and disorders of the neurological system. In certain instances, these neurologic disorders result from an increase or decrease in NMDA-receptor activity.

### BACKGROUND

Neurologic disorders are abnormal conditions of the nervous system. They can be categorized according to the structure or primary location within the nervous system affected, the nature of the dysfunction or the primary cause (e.g., genetic disorder, injury, infection). Some neurologic disorders, like depression, Parkinson's disease and stroke, are well-known while others are very rare. One recent study by the World Health Organization (WHO) found that neurologic disorders account for almost 11% of total global disease burden worldwide. Collectively, the burden of neurologic disorders is hard to overstate, and includes direct health care costs, disability, quality of life and lost productivity. For example, Alzheimer's disease alone drains more than $148 billion from the U.S. economy each year. The burden of neurologic disorder is expected to increase on a global basis, as demographic changes in the world's most populous countries will result in a significant increase in the number of persons with neurodegenerative diseases over the next few decades.

### NMDA Receptors

There are four classes of excitatory amino acids (EAA) receptors in brain that mediate neuronal activity: NMDA (N-methyl-D-aspartate), AMPA (2-amino-3-(methyl-3-hydroxyisoxazol-4-yl)propanoic acid), kainate and metabotropic receptors. Glutamate receptors mediate fast excitatory synaptic transmission in the central nervous system and are widely localized on neuronal and non-neuronal cells, regulating a broad spectrum of processes in the central and peripheral nervous system. The NMDA subtype of glutamate-gated ion channels mediates excitatory synaptic transmission between neurons in the central nervous system (Traynelis et al. Pharmacol Rev (2010) 62:405-96).

NMDA receptors are composed of GluN1, GluN2 (A, B, C, and D), and GluN3 (A and B) subunits, which determine the functional properties of native NMDA receptors. Co-expression of the GluN1 subunit with one or more GluN2 subunits is required to form functional channels. In addition to glutamate binding on GluN2 subunits, the NMDA receptor requires the binding of a co-agonist, glycine at GluN1 subunits, to allow the receptor to function. A glycine or D-serine binding site is also found on GluN3 subunits. At resting membrane potentials, NMDA receptors are largely inactive due to a voltage-dependent block of the channel pore by magnesium ions. Depolarization releases this channel block and permits passage of calcium and monovalent ions such as sodium ions.

NMDA receptors participate in a wide range of both physiological and pathological processes in the central nervous system and are found in neurons throughout the brain including the cortico-limbic regions which have been postulated to play a role in emotional functions, anxiety and depression (Tzschentke T M (2002) Amino Acids 23:147-152).

NMDA receptor antagonists can also be beneficial in the treatment of Parkinson's Disease (Blandini and Greenamyre (1998), Fundam Clin Pharmacol 12:4-12), brain cancers (Takano, T., et al. (2001), Nature Medicine 7:1010-1015; Rothstein, J. D. and Bren, H. (2001) Nature Medicine 7:994-995; Rzeski, W., et al. (2001), Proc. Nat'l Acad. Sci. 98:6372), and neuropsychiatric disorders including depressive disorders and bipolar disorders, which affect more than 60 million Americans each year.

NMDA receptor antagonists may also be beneficial in the treatment of chronic pain. For example, it has been reported that NMDA receptor antagonists produce an analgesic effect under certain conditions (Wong, et al. (1995) Acta Anaesthesiologica Sinica 33, 227-232). Nerve ligation, carrageenan-induced hyperalgesia, and wind-up pain in rats were all relieved by non-competitive, competitive, and GluN2B selective NMDA receptor antagonists (Boyce et al., (1999) Neuropharmacol 38: 611-623). Chronic pain, including neuropathic pain such as that due to injury of peripheral or central nerves, has often proved very difficult to treat.

U.S. Pat. No. 7,019,016 to Pfizer provides methods for treating certain disorders which comprise administration of certain GluN2B subunit selective NMDA antagonists. The disorders that can be treated by the invention include hearing loss, vision loss, neurodegeneration caused by epileptic seizures, neurotoxin poisoning, Restless Leg Syndrome, multi-system atrophy, non-vascular headache, and depression.

U.S. Pat. No. 5,710,168 claims the use of certain compounds having GluN2B subunit selectivity for treating a disease or condition which is susceptible to treatment by blocking of NMDA receptor sites, including traumatic brain injury, spinal cord trauma, pain, psychotic conditions, drug addiction, migraine, hypoglycemia, anxiolytic conditions, urinary incontinence, and ischemic events arising from CNS surgery, open heart surgery or any procedure during which the function of the cardiovascular system is compromised.

U.S. Pat. No. 6,479,553 to AstraZeneca provides certain compounds, in particular memantine, budipine, amantidine, 5-aminocarbonyl-10,11-dihydro-5H-dibenzo[a,d]cyclohepten-5,10-imine, dextromethorphan and NPS 1506, and the compounds disclosed in EP 279 937 and EP 633 879, specifically (S)-1-phenyl-2-(2-pyridyl)ethanamine as potentially useful as antidepressant agents. In particular, the compounds were expected to be useful in the treatment of depression associated with neurodegenerative disorders such as Alzheimer's disease.

U.S. Pat. No. 6,432,985 to Hoffman La-Roche provides certain neuroprotective substituted piperidine compounds with activity as NMDA GluN2B subtype selective antagonists

PCT Publication WO 06/017409 to Merck & Co. provides certain 1,3-disubstituted heteroaryl compounds are N-methyl-D-aspartate receptor antagonists useful for treating neurological condition e.g. pain, Parkinson's disease, Alzheimer's disease, anxiety, epilepsy and stroke.

PCT Publication WO 02/072542, to Emory University describes a class of pH-dependent NMDA receptor antagonists that exhibit pH sensitivity tested in vitro using an oocyte assay and in an experimental model of epilepsy.

PCT Publication WO 09/006,437, to Emory University and NeurOp, Inc., describes a class of pH sensitive NMDA antagonists for treatment of disorders including stroke, traumatic brain injury, neuropathic pain, epilepsy, and related neurologic events or neurodegeneration.

### pH Sensitive NMDA Receptors

The extracellular pH is highly dynamic in mammalian brain, and influences the function of a multitude of biochemical processes and proteins, including glutamate receptor function. pH changes are extensively documented in the central nervous system during synaptic transmission, glutamate receptor activation, glutamate receptor uptake, and prominently during pathological states such as ischemia and seizures (Siesjo, B K (1985), Progr Brain Res 63:121-154; Chesler, M (1990), Prog Neurobiol 34:401-427; Chesler and Kaila (1992), Trends Neurosci 15:396-402; Amato et al. (1994), J Neurophysiol 72:1686-1696). In addition to ischemia and seizures, there are various other examples of situations in which pH changes under normal and abnormal conditions, including neuropathic pain (Jendelova & Sykova (1991) Glia 4: 56-63; Chvatal et al. (1988) Physiol Bohemoslov 37: 203-212; Sykova et al. (1992) Can J Physiol Pharmacol 70: Suppl S301-309; Sykova & Svoboda (1990) Brain Res 512: 181-189) Parkinson's disease pH (see, for example, Chesler (1990) Prog Neurobiol 34: 401-427; Chesler & Kaila (1992) Tr Neurosci 15: 396-402; and Kaila & Chesler (1998) “Activity evoked changes in extracellular pH” in pH and Brain function (eds Kaila and Ransom). Wiley-Liss, New York), and various types of brain injury (Kaku et al. (1993), Science 260:1516-1518; Munir and McGonigle (1995), J Neurosci 15:7847-7860; Vornov et al. (1996), J Neurochem 67:2379-2389; Gray et al. (1997), J Neurosurg Anesthesiol 9:180-187; O'Donnell and Bickler (1994), Stroke 25:171-177; reviewed by Tombaugh and Sapolsky (1993), J Neurochem 61:793-803).

PCT Publication WO 06/023957 to Emory University describes processes for selection of a compound which may be useful in the treatment of an ischemic injury or a disorder that lowers the pH in a manner that activates the NMDA receptor antagonist.

There remains a need for improved treatments for neurological disorders, including neuropsychiatric disorders, neurodegenerative disorders and other neurological disorders of diverse origin, that are both safe and effective.

### SUMMARY

Compounds of Formula I, II, III, IV and V are provided for the treatment or prophylaxis of neurologic disorders including but not limited to neuropsychiatric and neurodegenerative disorders. In certain instances, the neurological disorders are known to result from an increase or decrease in NMDA receptor activity. In exemplary embodiments, compounds for use in the treatment of depression or anxiety in a host in need thereof. Certain compounds described herein have enhanced activity in brain tissue having lower-than-normal pH due to pathological conditions.

Pharmaceutical compositions of the Compounds of Formula I, II, III, IV and VI are also provided, comprising a Compounds of Formula I, II, III, IV and VI, alone or in combination, and a pharmaceutical carrier. Optionally, the pharmaceutical composition may comprise one or more excipients. In certain embodiments, the pharmaceutical composition may further comprise one or more active agents or materials, e.g., a therapeutic agent other than the Compounds of Formula I, II, III, IV and VI.

Methods of treating neurological disorders by administering a Compound of Formula I, II, III, IV and V, optionally in combination with a pharmaceutical carrier, to a host in need thereof are also provided. In exemplary embodiments, methods of treatment or prophylaxis of neuropsychiatric disorders, in particular depression and anxiety are provided comprising administering a compound according to the embodiments optionally in combination with a pharmaceutically acceptable carrier, to a host in need thereof.

In certain embodiments, the compounds are used for the treatment or prophylaxis of neuropsychiatric disorders, and in particular, mood disorders, for example depressive disorders and bipolar disorders. Depressive disorders include, for example, major depressive disorder, depression, atypical depression, melancholic depression, psychotic major depression, catatonic depression, postpartum depression, treatment-resistant depression, treatment-resistant bipolar depression, seasonal affective disorder (SAD), dysthymia, and depressive disorders not otherwise specified (DD-NOS) such as those disorders that are impairing but do not otherwise meet the criteria for a specific depressive disorder, such as recurrent brief depression, minor depressive disorders or minor depression. Bipolar disorder, or manic depression, or a mood disorder described by alternating periods of mania and depression, includes, for example bipolar I, which is distinguished by the presence of one or more manic episodes or mixed episodes with or without major depressive episodes; bipolar II, which consists of recurrent intermittent hypomanic and depressive episodes; cyclothymia, which consists of recurrent hypomanic and dysthymic episodes, but no full manic episodes or major depressive episodes; and bipolar disorder not otherwise specified (BD-NOS), which are disorders wherein a patient suffers from some symptoms in the bipolar spectrum (e.g. manic and depressive symptoms) but does not otherwise meet the criteria for a specific bipolar disorder diagnosis.

In certain embodiments, the compounds are used for the treatment or prophylaxis of a depressive disorder in a host diagnosed with the disorder. In certain other embodiments, the compounds are used for treatment or prophylaxis of a bipolar disorder in a host diagnosed with the disorder. The compounds can also be used for treatment or prophylaxis of depressive or manic episodes. The compounds can be provided on a seasonal basis, especially in a host who has been diagnosed or is at risk of SAD or of depression.

In certain other embodiments, the compounds are useful in the treatment or prophylaxis of a disorder associated with a physiological insult. The disorder can include depression or bipolar disorder associated with an injury or with aging.

In other embodiments, the compounds are used for the treatment or prophylaxis of neurodegenerative disorders, and in particular, Parkinson's disease, Alzheimer's disease, Huntington's disease and Amyotrophic lateral sclerosis (ALS).

In certain other embodiments, compounds are provided for the treatment or prophylaxis of a disorder associated with NMDA receptor activity. In exemplary embodiments, the compounds rare provided to modulate NMDA receptor activity, for example to modulation of NMDA receptor activity and thereby alleviates a disorder described herein.

In exemplary embodiments, the compounds are provided for treatment or prophylaxis of a disorder in which extracellular pH is reduced physiological pH. In certain embodiments, the compounds are for treatment of a disorder in which extracellular pH is reduced below about 7. In certain specific embodiments, extracellular pH in a specific brain region is reduced below about 6.9, 6.8, 6.7, 6.6, 6.5 or 6.4.

In certain embodiments, compounds are provided for the treatment or prophylaxis of stroke, transient ischemia, global ischemia and hypoxia.

In certain other embodiments, compounds are provided for the treatment of pain. In one embodiment, the compounds are used to treat neuropathic pain or inflammatory pain.

In other embodiments, compounds are provided for the treatment or prophylaxis of epilepsy, traumatic brain injury or spinal cord trauma.

In certain embodiments, the compounds are administered in combination or alternation with other compounds, in particular embodiments another compound useful in the treatment or prophylaxis of neuropsychiatric disorders.

### DETAILED DESCRIPTION

Compounds of Formulae I, II, III, IV and V are provided, as well as pharmaceutical compositions thereof. The compounds of the present invention, and pharmaceutical compositions comprising the same, are useful in the treatment of neurologic disorders. In certain instances, the disorders are known to result from NMDA receptor activity. Typically, these compounds act as NMDA receptor antagonists. In certain embodiments, the compounds are allosteric NMDA inhibitors. In particular, the compounds are provided for treatment or prophylaxis of neuropsychiatric disorders.

### DEFINITIONS

Whenever a term in the specification is identified as a range (i.e. C₁₋₄ alkyl), the range independently refers to each element of the range. As a non-limiting example, C₁₋₄ alkyl means, independently, C₁, C₂, C₃ or C₄ alkyl. Similarly, when one or more substituents are referred to as being “independently selected from” a group, this means that each substituent can be any element of that group, and any combination of these groups can be separated from the group. For example, if R¹ and R² can be independently selected from X, Y and Z, this separately includes the groups R¹ is X and R² is X; R¹ is X and R² is Y; R¹ is X and R² is Z; R¹ is Y and R² is X; R¹ is Y and R² is Y; R¹ is Y and R² is Z; R¹ is Z and R² is X; R¹ is Z and R² is Y; and R¹ is Z and R² is Z.

The term “alkyl” as used herein, unless otherwise specified, refers to a substituted or unsubstituted, saturated, straight or branched hydrocarbon, including but not limited to those of C₁ to C₁₀. Examples of alkyl groups are methyl, ethyl, propyl, isopropyl, butyl, secbutyl, isobutyl, tertbutyl, 1-methylbutyl, 1,1-dimethylpropyl, pentyl, isopentyl, neopentyl, hexyl, and isohexyl. Unless otherwise specified, the alkyl group can be unsubstituted or substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thio, sulfonyl, ester, carboxylic acid, amide, phosphonyl, phosphinyl, thioether, oxime, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991. In certain embodiments, alkyl may be optionally substituted by one or more fluoro, chloro, bromo, iodo, hydroxy, heterocyclic, heteroaryl, carboxy, alkoxy, nitro, NH₂, N(alkyl)₂, NH(alkyl), alkoxycarbonyl, —N(H or alkyl)C(O)(H or alkyl), —N(H or alkyl)C(O)N(H or alkyl)₂, —N(H or alkyl)C(O)O(H or alkyl), —OC(O)N(H or alkyl)₂, —S(O)ₙ—(H or alkyl), —C(O)—N(H or alkyl)₂, cyano, alkenyl, cycloalkyl, acyl, hydroxyalkyl, heterocyclic, heteroaryl, aryl, aminoalkyl, oxo, carboxyalkyl, —C(O)—NH₂, —C(O)—N(H)O(H or alkyl), —S(O)₂—NH₂, —S(O)ₙ—N(H or alkyl)₂ and/or —S(O)₂—N(H or alkyl)₂.

The term “cycloalkyl” as used herein, unless otherwise specified, refers to a substituted or unsubstituted, mono- or bicyclic hydrocarbon, including but not limited to those of C₃ to C₁₀. Examples of cycloalkyl groups are cyclopropyl, cyclobutyl, cyclopentyl, cyclopentyl, cyclohexyl, cycloheptyl, and cyclooctyl. Unless otherwise specified, the cycloalkyl group can be unsubstituted or substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thio, sulfonyl, ester, carboxylic acid, amide, phosphonyl, phosphinyl, thioether, oxime, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991. In certain embodiments, cycloalkyl may be optionally substituted by one or more fluoro, chloro, bromo, iodo, hydroxy, heterocyclic, heteroaryl, carboxy, alkoxy, nitro, NH₂, N(alkyl)₂, NH(alkyl), alkoxycarbonyl, —N(H or alkyl)C(O)(H or alkyl), —N(H or alkyl)C(O)N(H or alkyl)₂, —N(H or alkyl)C(O)O(H or alkyl), —OC(O)N(H or alkyl)₂, —S(O)ₙ—(H or alkyl), —C(O)—N(H or alkyl)₂, cyano, alkenyl, cycloalkyl, acyl, hydroxyalkyl, heterocyclic, heteroaryl, aryl, aminoalkyl, oxo, carboxyalkyl, —C(O)—NH₂, —C(O)—N(H)O(H or alkyl), —S(O)₂—NH₂, —S(O)ₙ—N(H or alkyl)₂ and/or —S(O)₂—N(H or alkyl)₂.

The term “halo” or “halogen,” refers to chloro, bromo, iodo, or fluoro.

The term “heteroaryl” or “heteroaromatic,” refers to an aromatic that includes at least one sulfur, oxygen, nitrogen or phosphorus in the aromatic ring. The term “heterocyclic” refers to a non-aromatic cyclic group wherein there is at least one heteroatom, such as oxygen, sulfur, nitrogen, or phosphorus in the ring. Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, pteridinyl, aziridines, thiazole, isothiazole, oxadiazole, thiazine, pyridine, pyrazine, piperazine, piperidine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic or heterocyclic group can be optionally substituted with one or more substituent selected from halogen, haloalkyl, alkyl, alkoxy, hydroxy, carboxyl derivatives, amido, amino, alkylamino, dialkylamino. The heteroaromatic can be partially or totally hydrogenated as desired. Nonlimiting examples include dihydropyridine and tetrahydrobenzimidazole. In some embodiment, the heteroaryl may be optionally substituted by one or more fluoro, chloro, bromo, iodo, hydroxy, heterocyclic, heteroaryl, carboxy, alkoxy, nitro, NH₂, N(alkyl)₂, NH(alkyl), alkoxycarbonyl, —N(H or alkyl)C(O)(H or alkyl), —N(H or alkyl)C(O)N(H or alkyl)₂, —N(H or alkyl)C(O)O(H or alkyl), —OC(O)N(H or alkyl)₂, —S(O)ₙ—(H or alkyl), —C(O)—N(H or alkyl)₂, cyano, alkenyl, cycloalkyl, acyl, hydroxyalkyl, heterocyclic, heteroaryl, aryl, aminoalkyl, oxo, carboxyalkyl, —C(O)—NH₂, —C(O)—N(H)O(H or alkyl), —S(O)₂—NH₂, —S(O)ₙ—N(H or alkyl)₂ and/or —S(O)₂—N(H or alkyl)₂. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-tolylsulfonyl.

The term “aryl,” unless otherwise specified, refers to a carbon based aromatic ring, including phenyl, biphenyl, or naphthyl. The aryl group can be optionally substituted with one or more moieties selected from the group consisting of hydroxyl, acyl, amino, halo, alkylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991. In certain embodiments, the aryl group is optionally substituted by one or more fluoro, chloro, bromo, iodo, hydroxy, heterocyclic, heteroaryl, carboxy, alkoxy, nitro, NH₂, N(alkyl)₂, NH(alkyl), alkoxycarbonyl, —N(H or alkyl)C(O)(H or alkyl), —N(H or alkyl)C(O)N(H or alkyl)₂, —N(H or alkyl)C(O)O(H or alkyl), —OC(O)N(H or alkyl)₂, —S(O)ₙ—(H or alkyl), —C(O)—N(H or alkyl)₂, cyano, alkenyl, cycloalkyl, acyl, hydroxyalkyl, heterocyclic, heteroaryl, aryl, aminoalkyl, oxo, carboxyalkyl, —C(O)—NH₂, —C(O)—N(H)O(H or alkyl), —S(O)₂—NH₂, —S(O)ₙ—N(H or alkyl)₂ and/or —S(O)₂—N(H or alkyl)₂.

The term “aralkyl,” unless otherwise specified, refers to an aryl group as defined above linked to the molecule through an alkyl group as defined above.

The term “alkaryl,” unless otherwise specified, refers to an alkyl group as defined above linked to the molecule through an aryl group as defined above. Other groups, such as acyloxyalkyl, alkoxyalkyl, alkoxycarbonyl, alkoxycarbonylalkyl, alkylaminoalkyl, alkylthioalkyl, amidoalkyl, aminoalkyl, carboxyalkyl, dialkylaminoalkyl, haloalkyl, heteroaralkyl, heterocyclicalkyl, hydroxyalkyl, sulfonamidoalkyl, sulfonylalkyl and thioalkyl are named in a similar manner.

The term “alkoxy,” unless otherwise specified, refers to a moiety of the structure —O-alkyl, wherein alkyl is as defined above.

The term “acyl,” refers to a group of the formula C(O)R′ or “alkyl-oxy”, wherein R′ is an alkyl, aryl, alkaryl or aralkyl group, or substituted alkyl, aryl, aralkyl or alkaryl.

The term “alkenyl” The term “alkenyl” means a monovalent, unbranched or branched hydrocarbon chain having one or more double bonds therein. The double bond of an alkenyl group can be unconjugated or conjugated to another unsaturated group. Suitable alkenyl groups include, but are not limited to (C₂-C₈)alkenyl groups, such as vinyl, allyl, butenyl, pentenyl, hexenyl, butadienyl, pentadienyl, hexadienyl, 2-ethylhexenyl,2-propyl-2-butenyl,4-(2-methyl-3-butene)-pentenyl. An alkenyl group can be unsubstituted or substituted with one or two suitable substituents.

The term “carbonyl” refers to a functional group composed of a carbon atom double-bonded to an oxygen atom: —C═O, Similarly, C(O) or C(═O) refers to a carbonyl group.

The term “amino” refers to —NH₂, —NH(alkyl) or —N(alkyl)₂.

The term “thio” indicates the presence of a sulfur group. The prefix thio- denotes that there is at least one extra sulfur atom added to the chemical. The prefix ‘thio-’ can also be placed before the name of a compound to mean that an oxygen atom in the compound has been replaced by a sulfur atom. Although typically the term “thiol” is used to indicate the presence of —SH, in instances in which the sulfur atom would be have improper valance a radical if the hydrogen is improperly designated, the terms ‘thio’ and ‘thiol’ are used interchangeably, unless otherwise indicated.

The term “amido” indicates a group (H or alkyl)-C(O)—NH—.

The term “carboxy” designates the terminal group —C(O)OH.

The term “sulfonyl” indicates an organic radical of the general formula (H or alkyl)-S(═O)₂—(H or alkyl′), where there are two double bonds between the sulfur and oxygen.

The term “pharmaceutically acceptable salt” refers to salts or complexes that retain the desired biological activity of the compounds of the present invention and exhibit minimal undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is H or alkyl and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The term “ester” refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl for example phenoxymethyl, aryl including phenyl optionally substituted with halogen, C₁ to C₄ alkyl or C₁ to C₄ alkoxy. The term “ester” may also refer to a sulfonate ester, for example alkyl or aralkyl including methanesulfonyl; or a mono-, di- or triphosphate ester.

Pharmaceutically acceptable “prodrugs” can refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated decacylated, phosphorylated or dephosphorylated to produce the active compounds.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

It should be understood that the various possible stereoisomers of the groups mentioned above and herein are within the meaning of the individual terms and examples, unless otherwise specified. As an illustrative example, “1-methyl-butyl” exists in both (R) and the (S) form, thus, both (R)-1-methyl-butyl and (S)-1-methyl-butyl is covered by the term “1-methyl-butyl”, unless otherwise specified.

As used herein, “treat” or “treatment” refers to or “treat” means any treatment of a disease or disorder in a host such as a mammal, including: (a) protecting against the disease or disorder, that is, causing the clinical symptoms not to develop; (b) inhibiting the disease or disorder, that is, arresting, ameliorating, reducing, or suppressing the development of clinical symptoms; and/or (c) relieving the disease or disorder, that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

### Compounds

In exemplary embodiments, compounds of Formula I are provided, or a pharmaceutically acceptable salt, ester, prodrug or derivative thereof, as well as methods of treatment or prophylaxis of neurologic disorders comprising administering the compound to a host in need thereof:

[chemical structure]

wherein:
each X is independently C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
k is 0, 1, 2, 3, 4, or 5;

### Z is CH or N;

L is —NR¹—(CR³R⁴)ₙ—NR²—, —(CR³R⁴)ₙ—NR²—, —O—(CR³R⁴)ₙ—NR²—,

[chemical structure]

wherein each R¹ and R² is independently H, C₁₋₆ alkyl, C₂₋₆ alkenyl, or C₆₋₁₂ aralkyl;
each R³ and R⁴ is independently H, C₁₋₆ alkyl, C₂₋₆ alkenyl, C₆₋₁₂ aralkyl, C₁₋₆ alkoxyl, C(═O)—C₁₋₆ alkyl, C₁₋₆ haloalkyl, hydroxyl, halo, nitro or cyano; or CR³R⁴ is C═O;
n is 1, 2, 3, or 4;

### M is —CH—CH(OH)— or —CH(CH₂OH)—;

### Y is

[chemical structure]

wherein R⁵ is H or C₁₋₆ alkyl.

In exemplary embodiments, compounds of Formula II are provided, or a pharmaceutically acceptable salt, ester, prodrug or derivative thereof, as well as methods of treatment or prophylaxis of neurologic disorders comprising administering the compound to a host in need thereof:

[chemical structure]

wherein:
each X is independently C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
k is 0, 1, 2, 3, 4, or 5;

### Z is CH or N;

L is —NR¹—(CR³R⁴)ₙ—NR²—, —(CR³R⁴)ₙ—NR²—, —O—(CR³R⁴)ₙ—NR²—,

[chemical structure]

wherein each R¹ and R² is independently H, C₁₋₆ alkyl, C₂₋₆ alkenyl, or C₆₋₁₂ aralkyl;
each R³ and R⁴ is independently H, C₁₋₆ alkyl, C₂₋₆ alkenyl, C₆₋₁₂ aralkyl, C₁₋₆ alkoxyl, C(═O)—C₁₋₆ alkyl, C₁₋₆ haloalkyl, hydroxyl, halo, nitro or cyano; or CR³R⁴ is C═O;
n is 1, 2, 3, or 4;

### Y is

[chemical structure]

wherein R⁵ is H or C₁₋₆ alkyl.

In exemplary embodiments, compounds of Formula III are provided, or a pharmaceutically acceptable salt, ester, prodrug or derivative thereof, as well as methods of treatment or prophylaxis of neurologic disorders comprising administering the compound to a host in need thereof:

[chemical structure]

wherein:
X is C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;

### Z is CH or N;

### Y is

[chemical structure]

wherein R⁵ is H or C₁₋₆ alkyl.

In exemplary embodiments, compounds of Formula IV are provided, or a pharmaceutically acceptable salt, ester, prodrug or derivative thereof, as well as methods of treatment or prophylaxis of neurologic disorders comprising administering the compound to a host in need thereof:

[chemical structure]

wherein:
each X is independently C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
k is 0, 1, 2, 3, 4, or 5;

### Z is CH or N;

L is —NR¹—(CR³R⁴)ₙ—NR²—, —(CR³R⁴)ₙ—NR²—, —O—(CR³R⁴)ₙ—NR²—,

[chemical structure]

wherein each R¹ and R² is independently H, C₁₋₆ alkyl, C₂₋₆ alkenyl, or C₆₋₁₂ aralkyl;
each R³ and R⁴ is independently H, C₁₋₆ alkyl, C₂₋₆ alkenyl, C₆₋₁₂ aralkyl, C₁₋₆ alkoxyl, C(═O)—C₁₋₆ alkyl, C₁₋₆ haloalkyl, hydroxyl, halo, nitro or cyano; or CR³R⁴ is C═O;
n is 1, 2, 3, or 4;

### Y is

[chemical structure]

wherein R⁵ is H or C₁₋₆ alkyl.

In exemplary embodiments, compounds of Formula V are provided, or a pharmaceutically acceptable salt, ester, prodrug or derivative thereof, as well as methods of treatment or prophylaxis of neurologic disorders comprising administering the compound to a host in need thereof:

[chemical structure]

wherein:
X is C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;

### Z is CH or N;

### Y is

[chemical structure]

wherein R⁵ is H or C₁₋₆ alkyl.

In exemplary embodiments, X is C₁₋₆ alkyl, for example, methyl, ethyl, n-propyl, isopropyl, n-butyl, sec-butyl, isobutyl, tert-butyl, 1-methylbutyl, 1,1-dimethylpropyl, pentyl, isopentyl, neopentyl, hexyl, or isohexyl. In exemplary embodiments, X is C₁₋₆ haloalkyl, for example trifluoromethyl. In exemplary embodiments, X is C₃₋₆ cycloalkyl, for example cyclopropyl, cyclobutyl, or cyclohexyl. In exemplary embodiments, X is halo, for example fluoro, bromo, chloro, or iodo.

In exemplary embodiments, k is 1. In exemplary embodiments, k is 2. In exemplary embodiment, k is 1 or 2, Z is CH and at least one X group is in the para position.

In exemplary embodiments, Z is CH. In exemplary embodiments, Z is N.

In exemplary embodiments, L is —NR¹—(CR³R⁴)ₙ—NR²—. In exemplary embodiments, L is —(CR³R⁴)ₙ—NR²—. In exemplary embodiments, L is —O—(CR³R⁴)ₙ—NR²—. In exemplary embodiments, L is

[chemical structure]

In exemplary embodiments, L is

[chemical structure]

In exemplary embodiments, L is

[chemical structure]

In exemplary embodiments, L is

[chemical structure]

In exemplary embodiments, L is

[chemical structure]

In exemplary embodiments, L is

[chemical structure]

In exemplary embodiments, n is 1. In exemplary embodiments, n is 2. In exemplary embodiments, n is 3. In exemplary embodiments, n is 4.

In exemplary embodiments, Y is

[chemical structure]

In exemplary embodiments, Y is

[chemical structure]

In exemplary embodiments, Y is

[chemical structure]

In exemplary embodiments, Y is

[chemical structure]

In exemplary embodiments, Y is

[chemical structure]

In exemplary embodiments, the compound is selected from the group consisting of:

[chemical structure]

[chemical structure]

[chemical structure]

and pharmaceutically acceptable salts, esters, prodrugs and derivatives thereof.

In exemplary embodiments, the compound is selected from the group consisting of:

[chemical structure]

and pharmaceutically acceptable salts, esters, prodrugs and derivatives thereof.

In exemplary embodiments, the OH group in M creates a stereogenic center. In one embodiment, the OH group is in the R configuration. In another embodiment, the OH group is in the S configuration.

### Enantiomers

In certain embodiments, the compounds are provided as enantiomers. In one embodiment, the compound is provided as an enantiomer or mixture of enantiomers. In a particular embodiment, the compound is present as a racemic mixture. The enantiomer can be named by the configuration at the chiral center, such as R or S. In certain embodiments, the compound is present as a racemic mixture of R- and S-enantiomers. In certain embodiments, the compound is present as a mixture of two enantiomers. In one embodiment, the mixture has an enantiomeric excess in R. In one embodiment, the mixture has an enantiomeric excess in S. In certain other embodiments, the compound is in an enantiomeric excess of the R- or S-enantiomer. The enantiomeric excess can be at least 51%, 55%, 60%, 65%, 70%, 75%, 80%, 85%, 90%, 95%, 98%, or 99% in the single enantiomer. The enantiomeric excess can be at least 51%, 55%, 60%, 65%, 70%, 75%, 80%, 85%, 90%, 95%, 98%, or 99% in the R enantiomer. The enantiomeric excess can be at least 51%, 55%, 60%, 65%, 70%, 75%, 80%, 85%, 90%, 95%, 98%, or 99% in the S enantiomer.

In other embodiments, the compound is substantially in the form of a single enantiomer. In some embodiments, the compound is present substantially in the form of the R enantiomer. In some embodiments, the compound is present substantially in the form of the S enantiomer. The phrase “substantially in the form of a single enantiomer” is intended to mean at least 70% in the form of a single enantiomer, for example at least about 70%, 75%, 80%, 85%, 90%, 95%, 98%, or 99% in either the R or S enantiomer.

The enantiomer can be named by the direction in which it rotates the plane of polarized light. If it rotates the light clockwise as seen by the viewer towards whom the light is traveling, the isomer can be labeled (+), or referred to as dextrorotary; and if it rotates the light counterclockwise, the isomer can be labeled (−) or referred to as levorotary. In certain embodiments, the compound is present as a racemic mixture of (+) and (−) isomers. In certain embodiments, the compound is present as a mixture of two isomers. In one embodiment, the mixture has an excess in (+). In one embodiment, the mixture has an excess in (−). In certain other embodiments, the compound is in an excess of the (+) or (−) isomer. The isomeric excess can be at least about 51%, 55%, 60%, 65%, 70%, 75%, 80%, 85%, 90%, 95%, 98%, or 99% in the (+) isomer. The enantiomeric excess can be at least about 51%, 55%, 60%, 65%, 70%, 75%, 80%, 85%, 90%, 95%, 98%, or 99% in the (−) isomer.

In other embodiments, the compound is substantially in the form of a single optical isomer. In some embodiments, the compound is present substantially in the form of the (+) isomer. In other embodiments, the compound is present substantially in the form of the (−) isomer. The phrase “substantially in the form of a single optical isomer” is intended to mean at least about 70% or more in the form of a single isomer, for example at least about 70%, 75%, 80%, 85%, 90%, 95%, 98%, or 99% of either the (+) or (−) isomer.

### Methods of Use

The compounds described herein can generally be used to treat, prevent or produce a reduction in symptoms of neurologic disorders, which includes abnormalities of the nervous system—either as such or in the form of a pharmaceutical composition, as discussed further below. T

These disorders can be characterized by primary location, dysfunction/abnormality or cause. Central nervous system disorders impact the brain or spinal cord, while peripheral nervous system disorders affect the nerves. Causes include, for example, genetic abnormalities, developmental abnormalities, injury, ischemia, or trauma, infection, cancer or diseases and disorders of the vasculature that supplies the nervous system, for example stroke. In certain instances, the neurologic disorder may be associated with NMDA receptor activation, for example activation of NMDA receptors including a GluN2B subunit. In exemplary embodiments, the neurologic disorder may be associated with an increase or decrease in NMDA receptor activity. In certain embodiments, the disorder is a disorder which can be treated, prevented of for which symptoms can be reduced by modulation of the NMDA receptor activity.

Disorders that can be treated, prevented or for which symptoms can be reduced include neuropsychiatric disorders, neurodegenerative disorders, as well as neurologic disorders including neuropathic pain, inflammatory pain, stroke, traumatic brain injury, epilepsy, transient ischemia, global ischemia, hypoxia, spinal cord trauma and other neurologic events or brain injuries.

In certain embodiments, the compounds are used for the treatment or prevention of neuropsychiatric disorders. These disorders include, without limitation, depression, anxiety, bipolar disorder, obsessive-compulsive disorder, alcohol and substance abuse, and attention-deficit hyperactivity disorder.

In certain other embodiments, the compounds are used for the treatment or prevention of neurodegenerative disorders. These disorders are typically characterized by gradual and progressive nervous system dysfunction due to loss of neuronal cells and neuronal tissue and include, without limitation, Alzheimer's disease, Parkinson's disease, Huntington's disease, Amyotrophic lateral sclerosis (ALS/Lou Gehrig's disease), Multiple Sclerosis, spinal muscular atrophy, spinal & bulbar muscular atrophy, familial spastic paraparesis, Machado Joseph disease Friedreich's ataxia and Lewy body disease.

In certain embodiments, a method of treatment a neurologic disorder are provided including administering a compound of the present embodiments, or a pharmaceutically acceptable salt, ester or prodrug thereof, to a host in need thereof. In certain embodiments, the disorder is associated with NMDA receptor activation. In certain embodiments, the disorder is associated with an increase or decrease in NMDA receptor activity. In one embodiment, the disorder is a neuropsychiatric disorder. In another embodiment, the disorder is a neurodegenerative disorder. In certain other embodiments, the disorder is neuropathic pain. In yet further embodiments, the disorder is an injury resulting from an ischemic event or neuropathic injury or infection.

In certain embodiments, methods are provided to prevent neurodegeneration in patients with Parkinson's, Alzheimer's, Huntington's chorea, ALS, and other neurodegenerative conditions.

In certain embodiments, a method of treatment or prevention of neurologic disorder, such as a neuropsychiatric or neurodegenerative disease or disorder or a disorder resulting from injury, trauma, infection or ischemia, in a host is provided including administering a compound of the present embodiments or a pharmaceutically acceptable salt, ester or prodrug thereof to the host, either alone or in combination, in which the host is suffering from a reduced pH in a region of the brain. In certain embodiments, a disorder has caused a region with a pH below about 7.6, 7.5, 7.4, 7.3, 7.2, 7.1, 7, 6.9, 6.8, 6.7, 6.6, 6.5 or 6.4. In certain embodiments, the reduced pH is due to pathological conditions such as hypoxia resulting from stroke, traumatic brain injury, global ischemia, such as global ischemia that may occur during cardiac surgery, hypoxia, including hypoxia that may occur following cessation of breathing, pre-eclampsia, spinal cord trauma, epilepsy, status epilepticus, neuropathic pain, inflammatory pain, chronic pain, vascular dementia and glioma tumors.

In one embodiment, methods are provided to attenuate the progression of an ischemic or excitotoxic cascade by administering a compound of the present embodiments. In addition, methods are provided to decrease infarct volume by administering a compound of the present embodiments. Still further, methods are provided to decrease behavioral deficits associated with an ischemic event by administering a compound of the present embodiments. In one embodiment, methods are provided to treat patients with ischemic injury or hypoxia, or prevent or treat the neuronal toxicity associated with ischemic injury or hypoxia, by administering a compound or composition described herein. In one particular embodiment, the ischemic injury is stroke. In another particular embodiment, the ischemic injury is vasospasm after subarachnoid hemorrhage. In other embodiments, the ischemic injury is selected from, but not limited to, one of the following: traumatic brain injury, cognitive deficit after bypass surgery, cognitive deficit after carotid angioplasty; and/or neonatal ischemia following hypothermic circulatory arrest.

Further, compounds selected according to the methods or processes described herein can be used prophylactically to prevent or protect against such neurologic or neuropathologic diseases, disorders or conditions, such as those described herein. In one embodiment, patients with a predisposition for an ischemic event, such as a genetic predisposition, can be treated prophylactically with the methods and compounds described herein. In another embodiment, patients at risk for or exhibiting vasospasms can be treated prophylactically with the methods and compounds described herein. In a further embodiment, patients undergoing cardiac bypass surgery can be treated prophylactically with the methods and compounds described herein. In one embodiment, the compounds of the present invention can be used as neuroprotectants.

In another embodiment, methods are provided to treat patients with neuropathic pain or related disorders by administering a compound or composition described herein. In certain embodiments, the neuropathic pain or related disorder can be selected from the group including, but not limited to: peripheral diabetic neuropathy, postherpetic neuralgia, complex regional pain syndromes, peripheral neuropathies, chemotherapy-induced neuropathic pain, cancer neuropathic pain, neuropathic low back pain, HIV neuropathic pain, trigeminal neuralgia, and/or central post-stroke pain. This dysfunction can be associated with common symptoms such as allodynia, hyperalgesia, intermittent abnormal sensations, and spontaneous, burning, shooting, stabbing, paroxysmal or electrical-sensations, paresthesias, hyperpathia and/or dysesthesias, which can also be treated by the compounds and methods described herein.

Further, the compounds and methods described herein can be used to treat neuropathic pain resulting from peripheral or central nervous system pathologic events, including, but not limited to trauma; ischemia; infections or endocrinologic disorders, including, but not limited to, diabetes mellitus, diabetic neurophathy, amyloidosis, amyloid polyneuropathy (primary and familial), neuropathies with monoclonal proteins, vasculitic neuropathy, HIV infection, herpes zoster—shingles and/or postherpetic neuralgia; neuropathy associated with Guillain-Barre syndrome; neuropathy associated with Fabry's disease; entrapment due to anatomic abnormalities; trigeminal and other CNS neuralgias; malignancies; inflammatory conditions or autoimmune disorders, including, but not limited to, demyelinating inflammatory disorders, rheumatoid arthritis, systemic lupus erythematosus, Sjogren's syndrome; and cryptogenic causes, including, but not limited to idiopathic distal small-fiber neuropathy. Other causes of neuropathic pain that can be treated according to the methods and compositions described herein include, but are not limited to, exposure to toxins or drugs (such as arsenic, thallium, alcohol, vincristine, cisplatinum and dideoxynucleosides), dietary or absorption abnormalities, immuno-globulinemias, hereditary abnormalities and amputations (including mastectomy). Neuropathic pain can also result from compression of nerve fibers, such as radiculopathies and carpal tunnel syndrome.

In a further embodiment, methods are provided to treat patients with neurodegenerative diseases by administering a compound selected according to the methods or processes described herein. These neurodegenerative disorders include, without limitation, Alzheimer's disease, Parkinson's disease, Huntington's disease, Amyotrophic lateral sclerosis (ALS/Lou Gehrig's disease), Multiple Sclerosis, spinal muscular atrophy, spinal and bulbar muscular atrophy, familial spastic paraparesis, Machado Joseph disease, Friedreich's ataxia and Lewy body disease. In one embodiment, the neurodegenerative disease can be Parkinson's disease. In another embodiment, the neurodegenerative disease can be Alzheimer's disease. In another embodiment, the neurodegenerative disease can be Huntington's disease and/or ALS.

In another embodiment, methods are provided to treat patients with brain tumors by administering a compound selected according to the methods or processes described herein. In some embodiments, the compounds are useful in the treatment of tumor growth. In certain embodiments, the compounds reduce tumor mass. In one embodiment, the compounds are useful in the treatment or prophylaxis of a neurologic event involving acidification of brain or spinal cord tissue. In another embodiment, the NMDA receptor antagonists of this invention are useful both in the treatment of stroke and head trauma, and for use as prophylactic agents for at risk patients. The acid generated by ischemic tissue during stroke is harnessed since the neuroprotective agents described herein are more potent at acidic pH. In this way side effects are minimized in unaffected tissue since drug at these sites are less potent. These compounds may be used to reduce the amount of neuronal death associated with stroke and head trauma. They may be given chronically to individuals with epilepsy or who are at risk for stroke or head trauma, preoperatively in high risk heart/brain surgery, etc., in order to lengthen the window of opportunity for subsequent therapy.

In addition, methods are provided to treat the following diseases or neurological conditions, including, but not limited to: chronic nerve injury, chronic pain syndromes, such as, but not limited to diabetic neuropathy, ischemia, ischemia following transient or permanent vessel occlusion, seizures, spreading depression, restless leg syndrome, hypocapnia, hypercapnia, diabetic ketoacidosis, fetal asphyxia, spinal cord injury, traumatic brain injury, status epilepticus, epilepsy, hypoxia, perinatal hypoxia, concussion, migraine, hypocapnia, hyperventilation, lactic acidosis, fetal asphyxia during parturition, brain gliomas, and/or retinopathies by administering a compound selected according to the methods or processes described herein.

In certain embodiments, the compounds are used for the treatment or prevention of neuropsychiatric disorders. Generally, these disorders are mental disturbances attributable to diseases of the nervous system. These disorders include depression, anxiety, bipolar disorder, obsessive-compulsive disorder, alcohol and substance abuse, and attention-deficit hyperactivity disorder. In particular embodiments, the disorders are neuropsychiatric mood disorders, non-limiting examples of which include depression, including major depression, treatment-resistant depression and treatment-resistant bipolar depression, bipolar disorders including cyclothymia (a mild form of bipolar disorder), affective disorders such as SAD (seasonal affective disorder) and mania (euphoric, hyperactive, over inflated ego, unrealistic optimism). In certain embodiments, the disorder is treatment-resistant depression or treatment-resistant bipolar depression. Neuropsychiatric disorders also include attention deficit disorders such as ADD or ADHD. In certain embodiments, a method of treatment a neuropsychiatric disorder is provided including administering a compound of the invention, alone or in combination to a host diagnosed with the disorder. Uses of the compounds in the treatment or manufacture of a medicament for such disorders are also provided.

In certain embodiments, the compounds are used for the treatment of depression in a host diagnosed with the disorder. In certain other embodiments, the compounds are used for treatment of a bipolar disorder in a host diagnosed with the disorder. The compounds can also be used to diminish the severity of depressive or manic episodes or prevent future episodes. In certain embodiments, methods of treating seasonal disorders is provided including administering the compound to a host at risk of suffering from a SAD. In particular, the compounds can be provided on a seasonal basis. In some embodiments, the host has been diagnosed as suffering from or is at risk for SAD or depression. In certain embodiments, the host is at risk of suffering from a mania. The mania can be characterized by euphoria, hyperactivity, over-inflated ego, or unrealistic optimism. In certain embodiments, the host is suffering from an attention deficit disorders, for example ADD or ADHD.

Depression, formally called major depression, major depressive disorder or clinical depression, is a medical illness that involves the mind and body. Most health professionals today consider depression a chronic illness that requires long-term treatment, much like diabetes or high blood pressure. Although some people experience only one episode of depression, most have repeated episodes of depression symptoms throughout their life. Depression is also a common feature of mental illness, whatever its nature and origin. In other instances, the host does not have a history of a major psychiatric disorder but has been diagnosed with suffering from at least one depressive episode. In other instances, the host has been diagnosed with bipolar disorder. The host may also have been diagnosed as suffering from panic attacks or anxiety.

In some instances, the host is not suffering from a chronic disorder but is at risk of a depressive episode, anxiety or a panic attack due to environmental circumstances. The compounds may be given prophylactically to prevent onset of such an episode. For instance, in certain instances the compounds can be provided to a host before a plane trip, a public speech, or other potential stressful even that could lead to an episode. In some embodiments, therefore, a method of prevention of a neuropsychiatric episode is provided, including administering a compound of the invention to a host at risk of suffering from such an episode. In some instances, the compounds are useful for treatment or prophylaxis of disorders such as depression or bipolar disorder associated with an injury or with aging.

In one embodiment, the compounds provided herein block the GluN2B-containing NMDA receptors, have varying activity, or may be selective, against receptors containing GluN2A, GluN2C, GluN2D, GluN3A and GluN3B. In one embodiment, the compounds are selective NMDA receptor blockers. In one embodiment, the compounds are NMDA receptor antagonists selective for one or more GluN2B-, GluN2A-, GluN2C-, GluN2D-, GluN3A-, or GluN3B-containing receptors that do not interact with other receptors or ion channels at therapeutic concentrations. In one embodiment, the compound is a selective GluN1/GluN2A NMDA receptor antagonist. In one embodiment, the compound is a selective GluN1/GluN2B NMDA receptor antagonist. In one particular embodiment, the compounds can bind to the GluN1 and/or GluN2B subunit of NMDA receptors. In another particular embodiment, the compounds are selective for the GluN2B-containing NMDA receptor. In one embodiment, the compound is not an NMDA receptor glutamate site antagonist. In another embodiment, the compound is not an NMDA receptor glycine site antagonist.

GluN2B-containing NMDA receptors may also be referred to as NR2B-containing NMDA receptors. Similarly, GluN2A is used interchangeably with NR2A, GluN2D with NR2D, GluN2C with NR²C, GluN3A with NR3A, and GluN3B with NR3B.

In certain embodiments, the compounds are administered to a host suffering from or at risk of suffering from age-related depression. The compounds can be administered prophylactically to a host over the age of 60, or over the age of 70, or over the age of 80 to prevent or reduce the severity of depressive episodes.

In certain embodiments, compounds of the present invention can be used to activate or stimulate the mTOR signaling pathway. In one embodiment, the compounds can be used to modulate mTOR activity in the brain, for example in the prefrontal cortex. Compounds which modulate or stimulate mTOR signaling may be useful in the treatment or prophylaxis of depression and other neuropsychiatric disorders.

In exemplary embodiments, the compounds can be used to treat traumatic brain injury, for example traumatic brain injury caused by a blast or a blast injury. In exemplary embodiments, the compounds can be used to treat a blast injury. In exemplary embodiments, the compounds can be used to treat a concussion. In exemplary embodiments, the neurologic disorder is a traumatic brain injury. In exemplary embodiments, the neurologic disorder is a blast injury. In exemplary embodiments, the neurologic condition is a concussion.

In one embodiment, the compounds may be used to in the treatment of schizophrenia. In another embodiment, the compounds may not be used to treat schizophrenia.

Uses of the compounds in the treatment or manufacture of a medicament for any disorder described herein are also provided.

### Side Effects

In an additional aspect of the methods and processes described herein, the compound does not exhibit substantial toxic and/or psychotomimetic side effects. Side effects associated with prior NMDAR blockers include, but are not limited to, agitation, hallucination, confusion, stupor, paranoia, delirium, psychotomimetic-like symptoms, rotarod impairment, amphetamine-like stereotyped behaviors, stereotypy, psychosis memory impairment, motor impairment, anxiolytic-like effects, increased blood pressure, decreased blood pressure, increased pulse, decreased pulse, hematological abnormalities, electrocardiogram (ECG) abnormalities, cardiac toxicity, heart palpitations, motor stimulation, psychomotor performance, mood changes, short-term memory deficits, long-term memory deficits, arousal, sedation, extrapyramidal side-effects, ventricular tachycardia. Lengthening of cardiac repolarisation, agitation, ataxia, cognitive deficits and/or schizophrenia-like symptoms.

The compounds selected or identified according to the processes and methods described herein generally avoid substantial side effects associated with other classes of NMDA receptor antagonists. In a particular embodiment, the compound has a therapeutic index equal to or greater than at least 2. In another embodiment, the compound is at least 10 times more selective for binding to an NMDA receptor than any other glutamate receptor. In a further additional or alternative embodiment, the compound has a therapeutic index equal to or greater than at least 2:1, 3:1, 4:1, 5:1, 6:1, 7:1, 8:1, 9:1, 10:1, 15:1, 20:1, 25:1, 30:1, 40:1, 50:1, 75:1, 100:1 or 1000:1. The therapeutic index can be defined as the ratio of the dose required to produce toxic or lethal effects to dose required to produce therapeutic responses. It can be the ratio between the median toxic dose (the dosage at which 50% of the group exhibits the adverse effect of the drug) and the median effective dose (the dosage at which 50% of the population respond to the drug in a specific manner). The higher the therapeutic index, the more safe the drug is considered to be. It simply indicates that it would take a higher dose to invoke a toxic response that it does to cause a beneficial effect.

In one embodiment, such compounds do not substantially exhibit the side effects associated with NMDA receptor antagonists of the glutamate site, such as selfotel, D-CPPene (SDZ EAA 494) and AR-R15896AR (ARL 15896AR), including, agitation, hallucination, confusion and stupor (Davis et al. (2000) Stroke 31(2):347-354; Diener et al. (2002), J Neurol 249(5):561-568); paranoia and delirium (Grotta et al. (1995), J Intern Med 237:89-94); psychotomimetic-like symptoms (Loscher et al. (1998), Neurosci Lett 240(1):33-36); poor therapeutic ratio (Dawson et al. (2001), Brain Res 892(2):344-350); amphetamine-like stereotyped behaviors (Potschka et al. (1999), Eur J Pharmacol 374(2):175-187). In another embodiment, such compounds do not exhibit the side effects associated with NMDA antagonists of the glycine site, such as HA-966, L-701,324, d-cycloserine, CGP-40116, and ACEA 1021, including significant memory impairment and motor impairment (Wlaz, P (1998), Brain Res Bull 46(6):535-540). In a still further embodiment, such compounds do not exhibit the side effects of NMDA high affinity receptor channel blockers, such as MK-801 and ketamine, including, psychosis-like effects (Hoffman, D C (1992), J Neural Transm Gen Sect 89:1-10); cognitive deficits (decrements in free recall, recognition memory, and attention; Malhotra et al (1996), Neuropsychopharmacology 14:301-307); schizophrenia-like symptoms (Krystal et al (1994), Arch Gen Psychiatry 51:199-214; Lahti et al. (2001), Neuropsychopharmacology 25:455-467), and hyperactivity and increased stereotypy (Ford et al (1989) Physiology and behavior 46: 755-758.

The side effect profile of compounds can be determined by any method known to those skilled in the art. In one embodiment, motor impairment can be measured by, for example, measuring locomotor activity and/or rotorod performance. Rotorod experiments involve measuring the duration that an animal can remain on an accelerating rod. In another embodiment, memory impairment can be assessed, for example, by using a passive avoidance paradigm; Sternberg memory scanning and paired words for short-term memory, or delayed free recall of pictures for long-term memory. In a further embodiment, anxiolytic-like effects can be measured, for example, in the elevated plus maze task. In other embodiments, cardiac function can be monitored, blood pressure and/or body temperature measured and/or electrocardiograms conducted to test for side effects. In other embodiments, psychomotor functions and arousal can be measured, for example by analyzing critical flicker fusion threshold, choice reaction time, and/or body sway. In other embodiments, mood can be assessed using, for example, self-ratings. In further embodiments, schizophrenic symptoms can be evaluated, for example, using the PANSS, BPRS, and CGI, side-effects were assessed by the HAS and the S/A scale.

In one embodiment, the compound does not exhibit substantial toxic side effects, such as, for example, motor impairment or cognitive impairment. In a particular embodiment, the compound has a therapeutic index equal to or greater than at least 2. In another embodiment, the compound is at least 10 times more selective for binding to an NMDA receptor than any other glutamate receptor.

In exemplary embodiments, the IC₅₀ value of the compound is in the range of about 0.001 to about 10 μM, about 0.005 to about 10 μM, about 0.01 to about 10 μM, about 0.01 to about 9 μM, about 0.01 to about 8 μM, about 0.01 to about 7 μM, about 0.01 to about 6 μM, about 0.01 to about 5 μM, about 0.001 to about 5 μM, about 0.005 to about 5 μM, about 0.01 to about 4 μM, about 0.01 to about 3 μM, about 0.01 to about 2 μM, about 0.01 to about 1 μM, about 0.05 to about 7 μM, about 0.05 to about 6 μM, about 0.05 to about 5 μM, about 0.05 to about 4 μM, about 0.05 to about 3 μM, about 0.05 to about 2 μM, about 0.05 to about 1 μM, about 0.05 to about 0.5 μM, about 0.1 to about 7 μM, about 0.1 to about 6 μM, about 0.1 to about 5 μM, about 0.1 to about 4 μM, about 0.1 to about 3 μM, about 0.1 to about 2 μM, about 0.1 to about 1 μM, about 0.1 to about 0.5 μM, about 0.1 to about 0.4 μM, about 0.1 to about 0.3 μM, or about 0.1 to about 0.2 μM.

In certain embodiments, the compound binds to hERG receptors at an IC₅₀ at least 10 times the IC₅₀ of inhibition of an NMDA receptor at either pH 6.9, pH 7.4, or 7.6. In certain embodiments, the compound binds adrenergic receptors, in particular α1-adrenergic receptors at an IC₅₀ at least 10 times the IC₅₀ of inhibition of an NMDA receptor at either pH 6.9, pH 7.4 or 7.6. In specific embodiments the ratio of IC₅₀'s between either hERG binding or adrenergic receptor binding and NMDA receptor antagonism at pH 6.9 or pH 7.4 is greater than 50, or greater than 100, or greater than 500.

Certain studies have indicated that pH may be altered in brains of individuals suffering from certain neuropsychiatric disorder (see e.g. Karolewicz, et al. (2004) J. Neurochem 91:1057-66. Xing, et al. (2002) Schizophr Res. 58:21-30.) A reduced brain pH can be harnessed to engage the pH dependent antagonism of the agents described herein. In this way side effects are minimized in unaffected tissue since drug at these sites are less active.

In particular embodiments, the compound is pH sensitive. In specific embodiments, the compound exhibits a potency boost of at least 2, 3, 4, 5, 6, 7, 8, or 9 when comparing the IC₅₀ at physiological pH versus the IC₅₀ diseased pH (i.e., (IC₅₀ at phys pH/IC₅₀ at diseased pH)).

In one embodiment, the compound has an IC₅₀ value of less than 5 μM at a pH of about 6 to about 9. In one embodiment, the compound has an IC₅₀ value of less than 5 μM at a pH of about 6.9, about 7.4, or about 7.6. In one embodiment, the compound has an IC₅₀ value of less than 5 μM at physiological pH. In one embodiment, the compound has an IC₅₀ value of less than 5 μM at ischemic pH.

In exemplary embodiments, the IC₅₀ value of the compound at about pH 7.6 is in the range of about 0.001 to about 3 μM, about 0.01 to about 3 μM, about 0.05 to about 3 μM, about 0.7 to about 2 μM, or about 0.7 to about 1.5 μM.

In exemplary embodiments, the IC₅₀ value of the compound at about pH 6.9 is in the range of about 0.001 to about 3 μM, about 0.01 to about 2 μM, about 0.01 to about 1 μM, about 0.01 to about 0.5 μM, or about 0.01 to about 0.4 μM.

In exemplary embodiments, the ratio of the IC₅₀ at pH 7.6 to the IC₅₀ at pH 6.9 of the compound is about 1.1 to about 10, about 1.1 to about 9, about 1.5 to about 8, or about 1.7 to about 7.

### Pharmaceutical Compositions

Mammals, and specifically humans, suffering from or at risk of neuropsychiatric disorders can be treated by either targeted or systemic administration, via oral, inhalation, topical, trans- or sub-mucosal, subcutaneous, parenteral, intramuscular, intravenous or transdermal administration of a composition comprising an effective amount of the compounds described herein or a pharmaceutically acceptable salt, ester or prodrug thereof, optionally in a pharmaceutically acceptable carrier.

The compounds (or pharmaceutical composition thereof, i.e., a pharmaceutical composition comprising the Compounds I, II, III, or IV, alone or in combination) is typically administered by oral administration. Alternatively, compounds can be administered by inhalation. In another embodiment, the compound is administered transdermally (for example via a slow release patch), or topically. In yet another embodiment, the compound is administered subcutaneously, intravenously, intraperitoneally, intramuscularly, parenterally, or submucosally. In any of these embodiments, the compound is administered in an effective dosage range to treat the target condition.

In one embodiment, the exemplary compounds can be administered orally. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

When the compound is administered orally in the form of a dosage unit such as a tablets, pills, capsules, troches and the like, these can contain any of the following ingredients, or compounds of a similar nature: a binder (such as microcrystalline cellulose, gum tragacanth or gelatin); an excipient (such as starch or lactose), a disintegrating agent (such as alginic acid, Primogel, or corn starch); a lubricant (such as magnesium stearate or Sterotes); a glidant (such as colloidal silicon dioxide); a sweetening agent (such as sucrose or saccharin); and/or a flavoring agent (such as peppermint, methyl salicylate, or orange flavoring). When the dosage unit form is a capsule, it can contain, in addition to material of the above type, a liquid carrier (such as a fatty oil). In addition, dosage unit forms can contain various other materials which modify the physical form of the dosage unit, for example, coatings of sugar, shellac, or other enteric agents.

The exemplary compounds can also be administered orally as a component of an elixir, suspension, syrup, wafer, chewing gum or the like. A syrup may contain, in addition to the active compounds, a sweetening agent (such as sucrose, saccharine, etc.) and preservatives, dyes and colorings and flavors.

The exemplary compounds may be also administered in specific, measured amounts in the form of an aqueous suspension by use of a pump spray bottle. The aqueous suspension compositions of the present invention may be prepared by admixing the compounds with water and other pharmaceutically acceptable excipients. The aqueous suspension compositions according to the present invention may contain, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phospate as well as mixtures of citrate and phosphate buffers; surfactants, e.g. Polysorbate 80; and antimicrobial preservatives, e.g., benzalkonium chloride, phenylethyl alcohol and potassium sorbate.

In exemplary embodiments, the compounds are in the form of an inhaled dosage, for example an aerosol suspension, a dry powder or liquid particle form. The compounds may be prepared for delivery as a nasal spray or in an inhaler, such as a metered dose inhaler. Pressurized metered-dose inhalers (“MDI”) generally deliver aerosolized particles suspended in chlorofluorocarbon propellants such as CFC-11, CFC-12, or the non-chlorofluorocarbons or alternate propellants such as the fluorocarbons, HFC-134A or HFC-227 with or without surfactants and suitable bridging agents. Dry-powder inhalers can also be used, either breath activated or delivered by air or gas pressure such as the dry-powder inhaler disclosed in the Schering Corporation International Patent Application No. PCT/US92/05225, published 7 Jan. 1993 as well as the Turbuhaler™ (available from Astra Pharmaceutical Products, Inc.) or the Rotahaler™ (available from Allen & Hanburys) which may be used to deliver the aerosolized particles as a finely milled powder in large aggregates either alone or in combination with some pharmaceutically acceptable carrier e.g. lactose; and nebulizers.

Solutions or suspensions used for parenteral, intradermal, subcutaneous, or topical application can include at least some of the following components: a sterile diluent (such as water for injection, saline solution, fixed oils, polyethylene glycols, glycerine, propylene glycol or other synthetic solvents); antibacterial agents (such as benzyl alcohol or methyl parabens); antioxidants (such as ascorbic acid or sodium bisulfite); chelating agents (such as ethylenediaminetetraacetic acid); buffers (such as acetates, citrates or phosphates); and/or agents for the adjustment of tonicity (such as sodium chloride or dextrose). The pH of the solution or suspension can be adjusted with acids or bases, such as hydrochloric acid or sodium hydroxide.

A parenteral preparation can be enclosed in ampoules, disposable syringes or multiple dose vials made of glass or plastic.

Suitable vehicles or carriers for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients, and stabilizers can be used to prepare topical compositions. Examples of thickening agents include petrolatum, beeswax, xanthan gum, or polyethylene, humectants such as sorbitol, emollients such as mineral oil, lanolin and its derivatives, or squalene.

If administered intravenously, carriers can be physiological saline, bacteriostatic water, Cremophor EL™ (BASF, Parsippany, N.J.) or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the compound is then introduced into the container. The container is then swirled by hand to free lipid material from the sides of the container and to disperse lipid aggregates, thereby forming the liposomal suspension.

### Dosing

The compound (or pharmaceutical composition comprising the same) is administered for a sufficient time period to alleviate the undesired symptoms and the clinical signs associated with the condition being treated. In one embodiment, the compounds are administered less than three times daily. In one embodiment, the compounds are administered in one or two doses daily. In one embodiment, the compounds are administered once daily. In some embodiments, the compounds are administered in a single oral dosage once a day.

The active compound is included in the pharmaceutically acceptable carrier or diluent in an amount sufficient to deliver to a patient a therapeutic amount of compound in vivo in the absence of serious toxic effects. An effective dose can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication.

Typical systemic dosages for the herein described conditions are those ranging from 0.01 mg/kg to 1500 mg/kg of body weight per day as a single daily dose or divided daily doses. Preferred dosages for the described conditions range from 0.5-1500 mg per day. A more particularly preferred dosage for the desired conditions ranges from 5-750 mg per day. Typical dosages can also range from 0.01 to 1500, 0.02 to 1000, 0.2 to 500, 0.02 to 200, 0.05 to 100, 0.05 to 50, 0.075 to 50, 0.1 to 50, 0.5 to 50, 1 to 50, 2 to 50, 5 to 50, 10 to 50, 25 to 50, 25 to 75, 25 to 100, 100 to 150, or 150 or more mg/kg/day, as a single daily dose or divided daily doses. In one embodiment, the daily dose is between 10 and 500 mg/day. In another embodiment, the dose is between about 10 and 400 mg/day, or between about 10 and 300 mg/day, or between about 20 and 300 mg/day, or between about 30 and 300 mg/day, or between about 40 and 300 mg/day, or between about 50 and 300 mg/day, or between about 60 and 300 mg/day, or between about 70 and 300 mg/day, or between about 80 and 300 mg/day, or between about 90 and 300 mg/day, or between about 100 and 300 mg/day, or about 200 mg/day. In one embodiment, the compounds are given in doses of between about 1 to about 5, about 5 to about 10, about 10 to about 25 or about 25 to about 50 mg/kg. Typical dosages for topical application are those ranging from 0.001 to 100% by weight of the active compound.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

### Combination Treatment

The compound can also be mixed with other active agents or materials which do not impair the desired action, or with materials that supplement the desired action. The active compounds can be administered in conjunction, i.e. combination or alternation, with medications used in the treatment or prevention neuropathic pain, stroke, traumatic brain injury, epilepsy, and other neurologic events or neurodegeneration resulting from NMDA receptor activation or with medications used in the treatment or prevention neuropsychiatric disorders, such as those in which NMDA receptor activation is involved. In certain embodiment, the compounds can be administered in conjunction (combination or alternation) with other medications used in treatment or prophylaxis of inflammatory conditions. In certain embodiments, the combination can be synergistic.

Emergency treatment for an ischemic stroke, particularly when the stroke is diagnosed within 3 hours of the start of symptoms, includes thrombolytic, or clot-dissolving, medications such as tissue plasminogen activator (t-PA). Other treatments of an ischemic stroke involve administering to the patient an antiplatelet medication (aspirin, clopidogrel, dipyridamole), or anticoagulant medication (warfarin), dependent on the cause. Dextrorphan, a pharmacologically active metabolite of the cough suppressant dextromethorphan, is an NMDAR antagonists studied in human stroke patients. Selfotel, a competitive NMDAR antagonist, was also tested in human patients, however treated patients trended toward higher mortality than within placebo-treated cohorts, and therefore, trials were stopped prematurely. A trial of another NMDA receptor antagonist, aptiganel HCl (Cerestat), was terminated. A large, 1367-patient, efficacy trial with the agent GV150526 was completed in 2000. (http://www.emedicine.com/neuro/topic488.htm, Lutsep & Clark “Neuroprotective Agents in Stroke”, Apr. 30, 2004).

In one embodiment, the compound is administered in combination or alternation with a compound useful for the treatment of neurological disorders. In certain embodiments, the compound is administered in combination or alternation with a compound useful for treatment of neuropsychiatric disorders, such as a selective serotonin reuptake inhibitor (SSRI), a serotonin and norepinephrine reuptake inhibitor (SNRI), norepinephrine and dopamine reuptake inhibitor (NDRI), combined reuptake inhibitor and receptor blocker, tetracyclic antidepressant, tricyclic antidepressants (TCAs) (although TCAs tend to have numerous and severe side effects), or a monoamine oxidase inhibitor (MAOI).

Electroconvulsive therapy (ECT) can also be used to treat depression in conjunction with administration of a compound of the invention. Non-traditional treatment options include vagus nerve stimulation, transcranial magnetic stimulation and deep brain stimulation.

SSRIs include fluoxetine (Prozac, Sarafem), paroxetine (Paxil), sertraline (Zoloft), citalopram (Celexa) and escitalopram (Lexapro). SSRIs that have been approved by the Food and Drug Administration specifically to treat depression are: Citalopram (Celexa), Escitalopram (Lexapro), Fluoxetine (Prozac, Prozac Weekly), Paroxetine (Paxil, Paxil CR) and Sertraline (Zoloft). SNRIs that have been approved by the Food and Drug Administration specifically to treat depression are: Duloxetine (Cymbalta) and Venlafaxine (Effexor, Effexor XR). The only NDRI that has been approved by the Food and Drug Administration specifically to treat depression is Bupropion (Wellbutrin, Wellbutrin SR, Wellbutrin XL). The only tetracyclic antidepressant that has been approved by the Food and Drug Administration specifically to treat depression is Mirtazapine (Remeron, Remeron SolTab). Other compounds approved for treatment of neuropsychiatric disorders include Anafranil (clomipramine HCl); Aventyl (nortriptyline HCl); Desyrel (trazodone HCl); Elavil (amitriptyline HCl); Limbitrol (chlordiazepoxide/amitriptyline); Ludiomil (Maprotiline HCl); Luvox (fluvoxamine maleate); Marplan (isocarboxazid); Nardil (phenelzine sulfate); Norpramin (desipramine HCl); Pamelor (nortriptyline HCl); Parnate (tranylcypromine sulfate); Pexeva (paroxetine mesylate); Prozac (fluoxetine HCl); Sarafem (fluoxetine HCl); Serzone (nefazodone HCl); Sinequan (doxepin HCl); Surmontil (trimipramine); Symbyax (olanzapine/fluoxetine); Tofranil (imipramine HCl); Tofranil-PM (impiramine pamoate); Triavil (Perphenaine/Amitriptyline); Vivactil (protriptyline HCl); Wellbutrin (bupropion HCl); and Zyban (bupropion HCl). Combined inhibitors and blockers that have been approved by the Food and Drug Administration specifically to treat depression are: Trazodone, Nefazodone and Maprotiline.

Tricyclic antidepressants (TCAs) inhibit the reabsorption (reuptake) of serotonin and norepinephrine. They were among the earliest of antidepressants, hitting the market in the 1960s, and they remained the first line of treatment for depression through the 1980s, before newer antidepressants arrived. TCAs that have been approved by the Food and Drug Administration specifically to treat depression are: Amitriptyline, Amoxapine, Desipramine (Norpramin), Doxepin (Sinequan), Imipramine (Tofranil), Nortriptyline (Pamelor), Protriptyline (Vivactil) and Trimipramine (Surmontil)

MAOIs that have been specifically approved by the Food and Drug Administration to treat depression are: Phenelzine (Nardil), Tranylcypromine (Parnate), Isocarboxazid (Marplan) and Selegiline (Emsam). Emsam is the first skin (transdermal) patch for depression.

Any of the compounds of the invention can be administered in combination with another active agent. In certain embodiments, the second active is one that is effective in treatment of a neuropsychiatric disorder. However, in certain other embodiments, the second active is one that is effective against an underlying disorder that is associated with a neuropsychiatric symptom. Examples of such disorders are heart disease, Alzheimer's disease and Parkinson's diseases. In certain embodiments, the compounds can be administered in combination in a single dosage form or injection, or administered concurrently. In other embodiments, the compounds are administered in alternation.

The following examples are provided to illustrate the present invention and are not intended to limit the scope thereof. Those skilled in the art will readily understand that known variations of the conditions and processes of the following preparative procedures can be used to manufacture the desired compounds. The materials required for the embodiments and the examples are known in the literature, readily commercially available, or can be made by known methods from the known starting materials by those skilled in the art.

### EXAMPLES

### Example 1

### General Synthesis of Exemplary Olefin and Comparative Keto Compounds

General procedures: All reagents were obtained from commercial suppliers and used without further purification. Unless otherwise specified, all reactions were carried out either in an atmosphere of nitrogen or argon gas. The reaction progress was monitored by thin layer chromatography (TLC) on precoated glass plates (silica gel HL UV254, 0.25 mm thickness) purchased from Sorbent Technologies. Purification was carried out by flash column chromatography with high performance silica gel (230-400 mesh) either manually or using Combiflash companion Teledyne Isco instrument. The ¹H NMR and ¹³C NMR spectra were recorded on a Varian or a Bruker 400 MHz spectrometer. The NMR data were obtained using deuterated chloroform (CDCl₃) or deuterated dimethylsulfoxide (DMSO-d₆) as solvents and referenced to the residual solvent peak; chemical shifts are reported in parts per million and coupling constants in hertz (Hz). Mass spectra were recorded on either a VG 70-S Nier Johnson or JEOL mass spectrometer. The purity of the compounds was evaluated by analytical HPLC using Shimadzu LC-20AT prominence liquid chromatography. Elemental analyses were performed at Atlantic Microlab (Norcross, Ga.) for C, H, and N.

The following is the general scheme and procedures for the preparation of exemplified compounds.

[chemical structure]

### Preparation of Compounds 3 and 4 (Scheme 1)

An ethanolic solution containing an equimolar (10 mmol) mixture of substituted aryl piperazine and vinyl epoxirane compounds was heated at 50° C. for 12 h. After cooling to room temperature, the volatiles were evaporated yielding a mixture of isomers as a white solid. The substance was purified by flash column chromatography on silica gel (230-400 mesh; 40 g) using gradient solvent mixture of either hexane:ethyl acetate (100:0 to 50:50) or dichloromethane:methanol (100:0 to 95:5) depending on the extent of separation of isomers. The yields of the pure isomers ranged from 10-40% for compound 3 and 10-20% for compound 4.

### Preparation of Compounds 6 and 7 (Scheme 1)

A mixture of bromo derivative 5 (1.6 mmol), potassium carbonate (4.9 mmol) and olefin intermediate 3 (1.6 mmol) in anhydrous DMF (50 mL) was degassed and filled with argon gas. Palladium catalyst (0.08 mmol) was added to the mixture and heated at 100° C. for 16 h. The resulting pale brown mixture was cooled to room temperature, which was diluted with brine and ethyl acetate followed by filtration to eliminate the insoluble material. A second method to process the reaction mixture was to filter through a pad of celite, and later mix with ethyl acetate and brine for extraction of products. The extracted organic layer was washed with brine, dried over sodium sulfate and evaporated on a rotary evaporator. The residue was purified by flash column chromatography on silica gel (230-400 mesh; 40 g) using a gradient solvent mixture of dichloromethane:methanol (100:0 to 95:5). In case of more polar compounds about 0.05% ammonia solution was used as another co-solvent to accelerate the elution. The less polar keto compound 6 was eluted first (Yield=5-20%) followed by olefin product 7 (Yield=5-60%).

### Preparation of Compound 8 (Scheme 1)

The procedure for preparation of olefin compound 8 was similar to compound 7 starting from the other olefin intermediate 4 and bromo derivative 5. The same palladium catalyst and base were used in this reaction. However, as the reaction proceeded slowly the heating period was longer (2-3 days) to obtain a reasonable yield. The isolated yields of product were in the range of 10-15%.

### Mass, NMR and HPLC Data of the Isolated Compounds

All the compounds were characterized by mass spectra and proton NMR. In few cases, ¹³C NMR was also studied to confirm the structures. In addition, the purity of final compounds was evaluated by HPLC studies using C18 column (5μ, 150×4.6 mm) with gradient water:acetonitrile containing 0.1% TFA as an eluting phase. The molecular structures of some of the isolated compounds and characterization data are as follows:

[chemical structure]

### (rac-) 1-(4-(4-propylphenyl)piperazin-1-yl)but-3-en-2-ol (9)

Yield: 0.45 g (16.4%)+1.1 g of isomer mixture. ¹H NMR (400 MHz, DMSO-d₆): δ 7.02 (d, 2H), 6.84 (d, 2H), 5.89 (m, 1H), 5.24 (d, 1H), 5.05 (d, 1H), 4.65 (br. s, 1H), 4.18 (br. s, 1H), 3.07 (br. s, 4H), 2.59 (br. s, 4H), 2.45 (t, 2H), 2.35 (br. s, 2H), 1.53 (dd, 2H), 0.87 (t, 3H).

[chemical structure]

### (rac-)(E)-6-(3-hydroxy-4-(4-(4-propylphenyl)piperazin-1-yl)but-1-en-1-yl)quinolin-2(1H)-one (10)

Yield: 0.22 g (33.6%). ¹H NMR (400 MHz, DMSO-d₆): δ 11.76 (s, 1H), 7.86 (d, 1H), 7.66 (s, 1H), 7.64 (d, 1H), 7.26 (d, 1H), 7.02 (d, 2H), 6.84 (d, 2H), 6.62 (d, 1H), 6.49 (d, 1H), 6.32 (dd, 1H), 4.84 (s, 1H), 4.38 (br. s, 1H), 3.08 (br. s, 4H), 2.61 (br. s, 4H), 2.42 (t, 2H), 1.51 (m, 2H), 0.87 (t, 3H). ES-MS m/z 418.2 (C₂₆H₃₁N₃O₂+1)⁺. HPLC data: purity 99.9%, retention time 8.1 min.

[chemical structure]

### (S,E)-6-(3-hydroxy-4-(4-(4-propylphenyl)piperazin-1-yl)but-1-en-1-yl)quinolin-2(1H)-one (11)

Yield: 0.03 g (6.6%)+0.2 g of impure product. The proton NMR and MS are same as reported for the racemic compound 10. HPLC data: purity 99%, retention time 8.1 min.

[chemical structure]

### (S)-1-(4-(4-cyclopropylphenyl)piperazin-1-yl)but-3-en-2-ol (12)

Yield: 0.33 g (46.2%). ¹H NMR (400 MHz, DMSO-d₆): δ 6.93 (d, 2H), 6.83 (d, 2H), 5.89 (m, 1H), 5.24 (d, 1H), 5.04 (m, 1H), 4.66 (d, 1H), 4.17 (m, 1H), 3.05 (m, 4H), 2.58 (m, 4H), 2.34 (m, 2H), 1.81 (m, 1H), 0.84 (m, 2H), 0.55 (m, 2H). ES-MS m/z 273.2 (C₁₇H₂₄N₂O+1)⁺.

[chemical structure]

### (S,E)-6-(4-(4-(4-cyclopropylphenyl)piperazin-1-yl)-3-hydroxybut-1-en-1-yl)quinolin-2(1H)-one (13)

Yield: 0.11 g (21.9%). ¹H NMR (400 MHz, DMSO-d₆): δ 11.76 (s, 1H), 7.88 (d, 1H), 7.65 (s, 1H), 7.63 (d, 1H), 7.26 (d, 1H), 6.93 (d, 2H), 6.82 (d, 2H), 6.62 (d, 1H), 6.51 (d, 1H), 6.32 (m, 1H), 4.84 (d, 1H), 4.37 (m, 1H), 3.06 (br. s, 4H), 2.62 (br. s, 4H), 2.44 (m, 2H), 1.81 (m, 1H), 0.85 (m, 2H), 0.54 (m, 2H). ES-MS m/z 416.2 (C₂₆H₂₉N₃O₂+1)⁺. HPLC data: purity 99%, retention time 7.8 min.

[chemical structure]

### 7-(3-oxo-4-(4-(4-(trifluoromethyl)phenyl)piperazin-1-yl)butyl)-2H-benzo[b][1,4]oxazin-3(4H)-one (14)

Yield: 0.030 g (6.72%). ¹H NMR (400 MHz, DMSO-d₆): δ 10.62 (s, 1H), 7.50 (d, 2H), 7.05 (d, 2H), 6.80 (m, 3H), 4.53 (s, 2H), 3.27 (s, 2H), 3.26 (m, 4H), 2.73 (dd, 4H), 2.52 (br. s, 4H). ¹H NMR (400 MHz, CDCl₃): δ 7.74 (br. s, 1H), 7.51 (d, 2H), 6.93 (d, 2H), 6.82 (m, 2H), 6.72 (d, 1H), 4.61 (s, 2H), 3.38 (br. s, 4H), 3.28 (br. s, 2H), 2.91 (m, 2H), 2.82 (m, 2H), 2.68 (br. s, 4H). ES-MS m/z 448.0 (C₂₃H₂₄F₃N₃O₃+1)⁺. HPLC data: purity 98%, retention time 7.68 min.

[chemical structure]

### (R,E)-7-(3-hydroxy-4-(4-(4-(trifluoromethyl)phenyl)piperazin-1-yl)but-1-en-1-yl)-2H-benzo[b][1,4]oxazin-3(4H)-one (15)

Yield: 0.060 g (13.4%)+0.065 g of impure product. ¹H NMR (400 MHz, DMSO-d₆): δ 10.73 (s, 1H), 7.50 (d, 2H), 7.04 (m, 4H), 6.84 (d, 1H), 6.49 (d, 1H), 6.22 (dd, 1H), 4.83 (s, 1H), 4.56 (s, 2H), 4.34 (s, 1H), 3.31 (br. s, 4H), 2.62 (s, 4H), 2.44 (s, 2H). ES-MS m/z 448.0 (C₂₃H₂₄F₃N₃O₃+1)⁺. HPLC data: purity 99%, retention time 7.59 min.

[chemical structure]

### (S,E)-7-(3-hydroxy-4-(4-(4-(trifluoromethyl)phenyl)piperazin-1-yl)but-1-en-1-yl)-2H-benzo[b][1,4]oxazin-3(4H)-one (16)

Yield: 0.065 g (14.5%)+0.11 g of impure product. ¹H NMR (400 MHz, DMSO-d₆): Same as the R— enantiomer 15. ES-MS m/z 448.3 (C₂₃H₂₄F₃N₃O₃+1)⁺. HPLC data: purity 99.8%, retention time 7.58 min.

[chemical structure]

### 6-(4-(4-(4-cyclopropylphenyl)piperazin-1-yl)-3-oxobutyl)-3,4-dihydroquinazolin-2(1H)-one (17)

Yield: 0.065 g (10.9%). ¹H NMR (400 MHz, DMSO-d₆): δ 8.91 (s, 1H), 6.91 (m, 4H), 6.80 (d, 2H), 6.79 (s, 1H), 6.67 (d, 1H), 4.25 (s, 2H), 3.20 (s, 2H), 3.03 (s, 4H), 2.71 (dd, 4H), 2.50 (s, 4H), 1.79 (br. s, 1H), 0.85 (m, 2H), 0.54 (s, 2H). ES-MS m/z 419.51 (C₂₅H₃₀N₄O₂+1)⁺. HPLC data: purity 98.4%, retention time 7.06 min.

[chemical structure]

### (R,E)-6-(4-(4-(4-cyclopropylphenyl)piperazin-1-yl)-3-hydroxybut-1-en-1-yl)-3,4-dihydroquinazolin-2(1H)-one (18)

Yield: 0.040 g (6.7%). ¹H NMR (400 MHz, DMSO-d₆): δ 9.04 (s, 1H), 7.15 (s, 2H), 6.92 (d, 2H), 6.81 (m, 3H), 6.71 (d, 1H), 6.46 (d, 1H), 6.13 (dd, 1H), 4.75 (s, 1H), 4.29 (m, 3H), 3.05 (s, 4H), 2.60 (s, 4H), 2.41 (s, 2H), 1.80 (s, 1H), 0.84 (d, 2H), 0.54 (s, 2H). ES-MS m/z 419.3 (C₂₅H₃₀N₄O₂+1)⁺. HPLC data: purity 98.05%, retention time 6.98 min.

[chemical structure]

### (rac)-1-(4-(4-Chlorophenyl)piperazin-1-yl)but-3-en-2-ol (19)

Yield: 1.46 g (53.8%). ¹H NMR (400 MHz, DMSO-d₆): δ 7.22 (d, 2H), 6.94 (d, 2H), 5.89 (m, 1H), 5.24 (d, 1H), 5.05 (d, 1H), 4.69 (d, 1H), 4.16 (t, 1H), 3.11 (m, 4H), 2.60 (m, 4H), 2.34 (m, 2H).

[chemical structure]

### 6-(4-(4-(4-Chlorophenyl)piperazin-1-yl)-3-oxobutyl)-3,4-dihydroquinolin-2(1H)-one (20)

Yield: 0.025 g (5.4%). ¹H NMR (400 MHz, DMSO-d₆): δ 9.99 (s, 1H), 7.34 (d, 2H), 7.20 (d, 2H), 6.96 (m, 4H), 6.75 (d, 1H), 3.22 (s, 2H), 3.11 (t, 4H), 2.82 (t, 2H), 2.77 (m, 4H), 2.55 (t, 4H), 2.39 (t, 2H). ES-MS m/z 412.1 (C₁₄H₁₉ClN₂O+1)⁺. HPLC data: purity 98%, retention time 7.6 min.

[chemical structure]

### (rac)-(E)-6-(4-(4-(4-Chlorophenyl)piperazin-1-yl)-3-hydroxybut-1-en-1-yl)-3,4-dihydroquinolin-2(1H)-one (21)

Yield: 0.025 g (5.4%). ¹H NMR (400 MHz, DMSO-d₆): δ 10.10 (s, 1H), 7.22 (m, 5H), 6.94 (d, 2H), 6.80 (d, 1H), 6.49 (d, 1H), 6.19 (dd, 1H), 4.79 (d, 1H), 4.33 (m, 1H), 3.13 (m, 4H), 2.86 (t, 2H), 2.61 (br. s, 4H), 2.39 (m, 2H). ES-MS m/z 412.1 (C₂₃H₂₆ClN₃O₂+1)⁺. HPLC data: purity 97%, retention time 7.3 min.

[chemical structure]

### (rac)-2-(4-(4-Chlorophenyl)piperazin-1-yl)but-3-en-1-ol (22)

Yield: 0.72 g (26.9%). ¹H NMR (400 MHz, DMSO-d₆): δ 7.22 (d, 2H), 6.92 (d, 2H), 5.78 (q, 1H), 5.22 (d, 1H), 5.18 (d, 1H), 4.40 (t, 1H), 3.59 (dt, 1H), 3.49 (dt, 1H), 3.11 (t, 4H), 2.91 (dd, 1H), 2.62 (m, 4H).

[chemical structure]

### (rac)-(E)-6-(3-(4-(4-chlorophenyl)piperazin-1-yl)-4-hydroxybut-1-en-1-yl)-3,4-dihydroquinolin-2(1H)-one (23)

Yield: 0.055 g (10.8%). ¹H NMR (400 MHz, DMSO-d₆): δ 10.11 (s, 1H), 7.30 (s, 1H), 7.20 (m, 3H), 6.92 (d, 2H), 6.80 (d, 1H), 6.45 (d, 1H), 6.14 (dd, 1H), 4.45 (t, 1H), 3.64 (dt, 1H), 3.56 (dt, 1H), 3.12 (br. s, 4H), 3.06 (m, 1H), 2.86 (t, 2H), 2.68 (m, 4H), 2.44 (t, 2H). ES-MS m/z 412.2 (C₂₃H₂₆ClN₃O₂+1)⁺. HPLC data: purity 93%, retention time 7.4 min.

[chemical structure]

### (S)-1-(4-(4-Isopropylphenyl)piperazin-1-yl)but-3-en-2-ol (24)

Yield: 0.50 g (51.0%). ¹H NMR (400 MHz, DMSO-d₆): δ 7.08 (d, 2H), 6.85 (d, 2H), 5.89 (m, 1H), 5.24 (d, 1H), 5.05 (d, 1H), 4.68 (d, 1H), 4.17 (m, 1H), 3.06 (t, 4H), 2.78 (dt, 1H), 2.58 (t, 4H), 2.35 (m, 2H).

[chemical structure]

### (S,E)-6-(3-Hydroxy-4-(4-(4-isopropylphenyl)piperazin-1-yl)but-1-en-1-yl)-3,4-dihydroquinolin-2(1H)-one (25)

Yield: 0.17 g (25.3%). ¹H NMR (400 MHz, DMSO-d₆): δ 10.10 (s, 1H), 7.25 (s, 1H), 7.19 (d, 1H), 7.07 (d, 2H), 6.85 (d, 2H), 6.80 (d, 1H), 6.50 (d, 1H), 6.18 (dd, 1H), 4.78 (d, 1H), 4.33 (br. s, 1H), 3.07 (br. s, 4H), 2.86 (t, 2H), 2.78 (dt, 1H), 2.61 (br. s, 4H), 2.43 (m, 4H), 1.16 (d, 6H). ES-MS m/z 420.2 (C₂₆H₃₃N₃O₂+1)⁺. HPLC data: purity 97%, retention time 7.7 min.

[chemical structure]

### (S)-1-(4-(4-Chlorophenyl)piperazin-1-yl)but-3-en-2-ol (26)

Yield: 0.48 g (50.4%). ¹H NMR (400 MHz, DMSO-d₆): Similar to compound 19.

[chemical structure]

### (S,E)-6-(4-(4-(4-Chlorophenyl)piperazin-1-yl)-3-hydroxybut-1-en-1-yl)-3,4-dihydro-quinolin-2(1H)-one (27)

Yield: 0.025 g (5.4%). ¹H NMR (400 MHz, DMSO-d₆): See compound 21. ES-MS m/z 412.2 (C₂₃H₂₆ClN₃O₂+1)⁺. HPLC data: purity 97%, retention time 7.4 min.

[chemical structure]

### (R)-1-(4-(4-Isopropylphenyl)piperazin-1-yl)but-3-en-2-ol (28)

Yield: 0.51 g (52.0%). ¹H NMR (400 MHz, DMSO-d₆): Similar to compound 24.

[chemical structure]

### (R,E)-6-(3-Hydroxy-4-(4-(4-isopropylphenyl)piperazin-1-yl)but-1-en-1-yl)-3,4-dihydroquinolin-2(1H)-one (29)

Yield: 0.17 g (25.3%). ¹H NMR (400 MHz, DMSO-d₆): δ 9.98 (s, 1H), 8.23 (d, 1H), 7.34 (d, 2H), 7.26 (d, 2H), 6.98 (s, 1H), 6.94 (d, 1H), 6.75 (d, 1H), 4.09 (m, 1H), 3.38 (br. s, 2H), 2.83 (t, 2H), 2.55 (m, 2H), 2.50 (m, 3H), 2.42 (t, 2H), 2.34 (m, 2H), 2.30 (m, 2H), 2.11 (m, 1H), 1.54 (m, 2H), 1.41 (m, 2H). ES-MS m/z 420.2 (C₂₆H₃₃N₃O₂+1)⁺. HPLC data: purity 97%, retention time 8.2 min.

[chemical structure]

### (R)-1-(4-(4-chlorophenyl)piperazin-1-yl)but-3-en-2-ol (30)

Yield: 0.50 g (52.5%). ¹H NMR (400 MHz, DMSO-d₆): Similar to compound 19.

[chemical structure]

### (R,E)-6-(4-(4-(4-Chlorophenyl)piperazin-1-yl)-3-hydroxybut-1-en-1-yl)-3,4-dihydroquinolin-2(1H)-one (31)

Yield: 0.070 g (9.1%). ¹H NMR (400 MHz, DMSO-d₆): Similar to compound 21. ES-MS m/z 412.2 (C₂₃H₂₆ClN₃O₂+1)⁺. HPLC data: purity 98%, retention time 7.4 min.

[chemical structure]

### (S)-1-(4-(4-(Trifluoromethyl)phenyl)piperazin-1-yl)but-3-en-2-ol (32)

Yield: 0.52 g (48.5%). ¹H NMR (400 MHz, CDCl₃): δ 7.51 (d, 2H), 6.95 (d, 2H), 5.83 (m, 1H), 5.39 (d, 1H), 5.21 (d, 1H), 4.25 (dt, 1H), 3.51 (br. s, 1H), 3.33 (m, 4H), 2.87 (m, 2H), 2.62 (m, 2H), 2.48 (m, 2H).

[chemical structure]

### (S,E)-6-(3-Hydroxy-4-(4-(4-(trifluoromethyl)phenyl)piperazin-1-yl)but-1-en-1-yl)-3,4-dihydroquinolin-2(1H)-one (33)

Yield: 0.100 g (13.2%). ¹H NMR (400 MHz, DMSO-d₆): δ 10.09 (s, 1H), 7.50 (d, 2H), 7.25 (s, 1H), 7.19 (d, 1H), 7.06 (d, 2H), 6.80 (d, 1H), 6.50 (d, 1H), 6.19 (dd, 1H), 4.80 (s, 1H), 4.34 (br. s, 1H), 3.28 (s, 4H), 2.86 (t, 2H), 2.62 (s, 4H), 2.44 (t, 4H). ES-MS m/z 446.2 (C₂₄H₂₆F₃N₃O₂+1)⁺. HPLC data: purity 98%, retention time 7.8 min.

[chemical structure]

### (R)-1-(4-(4-(Trifluoromethyl)phenyl)piperazin-1-yl)but-3-en-2-ol (34)

Yield: 0.60 g (60.0%). ¹H NMR (400 MHz, DMSO-d₆): δ 7.50 (d, 2H), 7.05 (d, 2H), 5.89 (m, 1H), 5.24 (d, 1H), 5.05 (d, 1H), 4.70 (d, 1H), 4.19 (br. s, 1H), 3.27 (s, 4H), 2.59 (s, 4H), 2.36 (m, 2H).

[chemical structure]

### (R,E)-6-(3-Hydroxy-4-(4-(4-(trifluoromethyl)phenyl)piperazin-1-yl)but-1-en-1-yl)-3,4-dihydroquinolin-2(1H)-one (35)

Yield: 0.20 g (24.5%). ¹H NMR (400 MHz, DMSO-d₆): Similar to compound 33. ES-MS m/z 446.8 (C₂₄H₂₆F₃N₃O₂+1)⁺. HPLC data: purity 98%, retention time 7.7 min.

[chemical structure]

### (S,E)-1-(4-(3-Hydroxy-4-(4-(4-isopropylphenyl)piperazin-1-yl)but-1-en-1-yl)phenyl)urea (36)

Yield: 0.048 g (16.1%). ¹H NMR (400 MHz, DMSO-d₆): δ 8.55 (s, 1H), 7.35 (d, 2H), 7.27 (d, 2H), 7.08 (d, 2H), 6.85 (d, 2H), 6.49 (d, 1H), 6.15 (dd, 1H), 5.84 (s, 2H), 4.75 (d, 1H), 4.33 (br. s, 1H), 3.08 (s, 4H), 2.79 (dd, 1H), 2.62 (s, 4H), 2.44 (m, 2H). ES-MS m/z 409.2 (C₂₄H₃₂N₄O₂+1)⁺. HPLC data: purity 98%, retention time 7.8 min.

[chemical structure]

### 1-(4-(3-Oxo-4-(4-(4-(trifluoromethyl)phenyl)piperazin-1-yl)butyl)phenyl)urea (37)

Yield: 0.056 g (12.9%). ¹H NMR (400 MHz, DMSO-d₆): δ 8.41 (s, 1H), 7.50 (d, 2H), 7.28 (d, 2H), 7.05 (d, 4H), 5.78 (s, 2H), 3.25 (m, 6H), 2.73 (t, 4H), 2.51 (s, 4H). ES-MS m/z 435.1 (C₂₂H₂₅F₃N₄O₂+1)⁺. HPLC data: purity 94%, retention time 7.4 min.

[chemical structure]

### (R,E)-1-(4-(3-Hydroxy-4-(4-(4-(trifluoromethyl)phenyl)piperazin-1-yl)but-1-en-1-yl)phenyl)urea (38)

Yield: 0.069 g (15.9%). ¹H NMR (400 MHz, DMSO-d₆): δ 8.55 (s, 1H), 7.50 (d, 2H), 7.36 (d, 2H), 7.28 (d, 2H), 7.06 (d, 2H), 6.50 (d, 1H), 6.15 (dd, 1H), 5.84 (s, 2H), 4.79 (d, 1H), 4.34 (br. s, 1H), 3.28 (s, 4H), 2.62 (s, 4H), 2.44 (s, 2H). ES-MS m/z 435.1 (C₂₂H₂₅F₃N₄O₂+1)⁺. HPLC data: purity 97%, retention time 7.4 min.

[chemical structure]

### (S,E)-1-(4-(3-Hydroxy-4-(4-(4-(trifluoromethyl)phenyl)piperazin-1-yl)but-1-en-1-yl)phenyl)urea (39) (Change data)

Yield: 0.056 g (12.9%). ¹H NMR (400 MHz, DMSO-d₆): Similar to compound 38. ES-MS m/z 435.4 (C₂₂H₂₅F₃N₄O₂+1)⁺. HPLC data: purity 96%, retention time 7.1 min.

[chemical structure]

### (R,E)-6-(3-hydroxy-4-(4-(4-(trifluoromethyl)phenyl)piperazin-1-yl)but-1-en-1-yl)-3,4-dihydroquinazolin-2(1H)-one (41)

Yield: 0.075 g (16.8%). ¹H NMR (400 MHz, DMSO-d₆): δ 9.05 (s, 1H), 7.50 (d, 2H), 7.17 (d, 2H), 7.06 (d, 2H), 6.80 (s, 1H), 6.71 (dd, 1H), 6.47 (d, 1H), 6.14 (dd, 1H), 4.09 (m, 1H), 4.78 (s, 1H), 4.30 (br. s, 3H), 3.27 (s, 4H), 2.61 (s, 4H), 2.43 (t, 2H). ES-MS m/z 447.2 (C₂₃H₂₅F₃N₄O₂+1)⁺. HPLC data: purity 97%, retention time 7.1 min.

[chemical structure]

### (S)-2-(4-(4-(Trifluoromethyl)phenyl)piperazin-1-yl)but-3-en-1-ol (42)

Yield: 1.0 g (13.9%). ¹H NMR (400 MHz, DMSO-d₆): δ 7.49 (d, 2H), 7.04 (d, 2H), 5.78 (ddd, 1H), 5.20 (m, 2H), 4.43 (d, 1H), 3.60 (m, 1H), 3.48 (m, 1H), 3.25 (t, 4H), 2.93 (dd, 1H), 2.63 (m, 4H).

[chemical structure]

### (S,E)-6-(4-Hydroxy-3-(4-(4-(trifluoromethyl)phenyl)piperazin-1-yl)but-1-en-1-yl)-3,4-dihydroquinolin-2(1H)-one (43)

Yield: 0.080 g (10.8%). ¹H NMR (400 MHz, DMSO-d₆): δ 10.11 (s, 1H), 7.49 (d, 2H), 7.30 (s, 1H), 7.20 (d, 2H), 7.04 (d, 2H), 6.80 (d, 1H), 6.46 (d, 1H), 6.14 (dd, 1H), 4.47 (br. s, 1H), 3.65 (m, 1H), 3.57 (m, 1H), 3.27 (s, 4H), 3.07 (dd, 1H), 2.86 (t, 2H), 2.68 (s, 4H), 2.44 (t, 2H). ES-MS m/z 446.2 (C₂₄H₂₆F₃N₃O₂+1)⁺. HPLC data: purity 97%, retention time 7.6 min.

[chemical structure]

### ((R)-1-(4-(5-(Trifluoromethyl)pyridin-2-yl)piperazin-1-yl)but-3-en-2-ol (44)

Yield: 0.34 g (31.6%). ¹H NMR (400 MHz, CDCl₃): δ 8.39 (s, 1H), 7.78 (d, 1H), 6.95 (d, 1H), 5.88 (br. s, 1H), 5.23 (d, 1H), 5.06 (d, 1H), 4.70 (s, 1H), 4.18 (s, 1H), 3.61 (s, 4H), 2.34 (br. s, 6H).

[chemical structure]

### (R,E)-6-(3-Hydroxy-4-(4-(5-(trifluoromethyl)pyridin-2-yl)piperazin-1-yl)but-1-en-1-yl)-3,4-dihydroquinolin-2(1H)-one (45)

Yield: 0.130 g (21.9%). ¹H NMR (400 MHz, DMSO-d₆): δ 10.09 (s, 1H), 8.40 (s, 1H), 7.78 (d, 1H), 7.25 (s, 1H), 7.18 (d, 1H), 6.95 (d, 1H), 6.80 (d, 1H), 6.49 (d, 1H), 6.18 (dd, 1H), 4.81 (d, 1H), 4.34 (s, 1H), 3.62 (s, 4H), 2.50 (s, 2H), 2.44 (t, 4H), 2.44 (t, 4H). ES-MS m/z 447.3 (C₂₃H₂₅F₃N₄O₂+1)⁺. HPLC data: purity 99%, retention time 7.0 min.

### Example 2

### Determination of IC₅₀ Values of GluN1/GluN2B Receptors Expressed in Xenopus oocytes in Human or Rat

Stage V and VI oocytes were surgically removed from the ovaries of large, well-fed and healthy Xenopus laevis anesthetized with 3-amino-benzoic acid ethyl ester (3 gm/l). Oocytes were injected within 24 hrs of isolation with 3-5 ng of GluN1 subunit cRNA and 7-10 ng of GluN2 cRNA subunit in a 50 nl volume, and incubated in Barth's solution at 18° C. for 2-7 d. Glass injection pipettes had tip sizes ranging from 10-20 microns, and were backfilled with mineral oil.

Two electrode voltage-clamp recordings were made 2-7 days post-injection. Oocytes were placed in a dual-track plexiglass recording chamber and dual recordings were made at room temperature (23° C.) using two Warner OC725C two-electrode voltage clamp amplifiers, arranged as recommended by the manufacturer. Glass microelectrodes (1-10 Megaohms) were filled with 300 mM KCl (voltage electrode) or 3 M KCl (current electrode). The bath clamps communicated across silver chloride wires placed into each side of the recording chamber, both were assumed to be at a reference potential of 0 mV. Oocytes were perfused with a solution comprised of (in mM) 90 NaCl, 1 KCl, 10 HEPES, 10 EDTA and 0.5 BaCl₂; pH was adjusted by addition of 1-3 M NaOH or HCl. Oocytes were recorded under voltage clamp at −40 mV. Final concentrations for glutamate and glycine were 50 μM and 30 μM, respectively. Concentration-response curves for experimental compounds were obtained by applying maximal glutamate/glycine, followed by glutamate/glycine plus variable concentrations of experimental compounds. Dose response curves consisting of 4 to 6 concentrations were obtained in this manner. The baseline leak current at −40 mV was measured before and after recording, and the full recording linearly corrected for any change in leak current. The level of inhibition by applied experimental compounds measured after a 6 min exposure to a compound was expressed as a percent of the initial glutamate response, and averaged together across oocytes from multiple experiments. Results were pooled, and the average percent responses at antagonist concentrations were fit by the equation,

Percent Response=(100−minimum)/(1+([conc]/IC₅₀)^{nH})+minimum

where minimum is the residual percent response in saturating concentration of the experimental compounds, IC₅₀ is the concentration of antagonist that causes half of the achievable inhibition, and nH is a slope factor describing steepness of the inhibition curve. Minimum was constrained to be between zero and 20.

Recordings were made at pH 6.9, pH 7.4, and pH 7.6.

TABLE 1
IC₅₀ data for Exemplary Compounds

| | pH 7.6 | | | | pH 6.9 | | | | IC₅₀ |
|---|---|---|---|---|---|---|---|---|---|
| Cpd | IC₅₀ | | | No. of | IC₅₀ | | | No. of | Ratio |
| No. | (μM) | nH | Ymin | Oocytes | (μM) | nH | Ymin | Oocytes | (7.6/6.9) |
| 10 | 0.177 | −1.24 | 20.0 | 14 | 0.084 | −0.90 | 0.0 | 15 | 2.1 |
| 11 | 0.086 | −0.52 | 10.0 | 10 | 0.050 | −0.87 | 10.0 | 11 | 1.7 |
| 13 | 0.142 | −0.80 | 10.0 | 12 | 0.063 | −0.81 | 10.0 | 12 | 2.3 |
| 15 | 1.1 | −1.04 | 20.0 | 32 | 0.246 | −0.88 | 9.1 | 32 | 4.5 |
| 16 | 0.943 | −1.29 | 4.6 | 17 | 0.248 | −0.67 | 0.0 | 20 | 3.8 |
| 18 | 0.177 | −1.28 | 13.1 | 16 | 0.055 | −0.78 | 0.0 | 18 | 3.2 |
| 21 | 0.278 | −1.28 | 20.0 | 17 | 0.142 | −0.81 | 0.0 | 19 | 2.0 |
| 23 | 1.40 | −0.60 | 0.0 | 13 | 0.321 | −1.09 | 10.0 | 12 | 4.4 |
| 25 | 0.271 | −1.22 | 7.4 | 12 | 0.090 | −0.61 | 0.0 | 16 | 3.0 |
| 27 | 0.700 | −0.64 | 0.0 | 12 | 0.227 | −0.95 | 0.0 | 13 | 3.1 |
| 29 | 0.651 | −1.31 | 10.0 | 12 | 0.240 | −0.54 | 0.0 | 15 | 2.7 |
| 31 | 0.429 | −0.90 | 0.0 | 12 | 0.129 | −0.64 | 10.0 | 12 | 3.3 |
| 33 | 0.817 | −0.65 | 0.0 | 12 | 0.189 | −0.56 | 0.0 | 15 | 4.3 |
| 35 | 0.391 | −0.75 | 5.0 | 28 | 0.073 | −0.68 | 5.0 | 26 | 5.4 |
| 36 | 0.146 | −1.05 | 4.4 | 25 | 0.062 | −0.82 | 2.7 | 28 | 2.4 |
| 38 | 0.517 | −0.69 | 10.0 | 47 | 0.084 | −0.64 | 6.7 | 47 | 6.2 |
| 39 | 0.453 | −0.69 | 20.0 | 20 | 0.148 | −0.43 | 0.0 | 19 | 3.1 |
| 41 | 0.078 | −1.04 | 5.7 | 40 | 0.019 | −0.64 | 0.0 | 33 | 4.2 |
| 43 | 0.993 | −1.09 | 5.7 | 34 | 0.173 | −0.67 | 2.7 | 36 | 5.7 |
| 45 | 1.1 | −0.80 | 20.0 | 28 | 0.174 | −0.80 | 15.5 | 39 | 6.3 |

TABLE 2
IC₅₀ data for Comparative Keto Compounds

| | |
|---|---|
| pH 7.6 | pH 6.9 |

| Compound | IC₅₀ | No. of | IC₅₀ | No. of |
|---|---|---|---|---|
| No. | (μM) | Oocytes | (μM) | Oocytes |
| 14 | >10* | 4 | ~7.8 | 7 |
| 17 | ~2.9 | 4 | ~1.3 | 2 |
| 20 | ~2.1 | 4 | ~2.3 | 2 |
| 37 | ~4.2 | 4 | ~8.7 | 6 |

We claim:

1. A compound of Formula I, or a pharmaceutically acceptable salt, ester, prodrug or derivative thereof: [chemical structure]
wherein:
each X is independently C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
k is 0, 1, 2, 3, 4, or 5;
Z is CH or N;
L is —NR¹—(CR³R⁴)ₙ—NR²—, —(CR³R⁴)ₙ—NR²—, —O—(CR³R⁴)ₙ—NR²—, [chemical structure]
wherein each R¹ and R² is independently H, C₁₋₆ alkyl, C₂₋₆ alkenyl, or C₆₋₁₂ aralkyl;
each R³ and R⁴ is independently H, C₁₋₆ alkyl, C₂₋₆ alkenyl, C₆₋₁₂ aralkyl, C₁₋₆ alkoxyl, C(═O)—C₁₋₆ alkyl, C₁₋₆ haloalkyl, hydroxyl, halo, nitro or cyano; or CR³R⁴ is C═O;
n is 1, 2, 3, or 4;
M is —CH—CH(OH)— or —CH(CH₂OH)—;
Y is [chemical structure]
wherein R⁵ is H or C₁₋₆ alkyl.

2. The compound of claim 1, wherein the compound is a compound of Formula II or a pharmaceutically acceptable salt, ester, prodrug or derivative thereof: [chemical structure]
wherein:
each X is independently C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
k is 0, 1, 2, 3, 4, or 5;
Z is CH or N;
L is —NR¹—(CR³R⁴)ₙ—NR²—, —(CR³R⁴)ₙ—NR²—, —O—(CR³R⁴)ₙ—NR²—, [chemical structure]
wherein each R¹ and R² is independently H, C₁₋₆ alkyl, C₂₋₆ alkenyl, or C₆₋₁₂ aralkyl;
each R³ and R⁴ is independently H, C₁₋₆ alkyl, C₂₋₆ alkenyl, C₆₋₁₂ aralkyl, C₁₋₆ alkoxyl, C(═O)—C₁₋₆ alkyl, C₁₋₆ haloalkyl, hydroxyl, halo, nitro or cyano; or CR³R⁴ is C═O;
n is 1, 2, 3, or 4;
Y is [chemical structure]
wherein R⁵ is H or C₁₋₆ alkyl.

3. The compound of claim 1, wherein the compound is a compound of Formula III or a pharmaceutically acceptable salt, ester, prodrug or derivative thereof: [chemical structure]
wherein:
X is C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
Z is CH or N;
Y is [chemical structure]
wherein R⁵ is H or C₁₋₆ alkyl.

4. The compound of claim 1, wherein the compound is a compound of Formula IV or a pharmaceutically acceptable salt, ester, prodrug or derivative thereof: [chemical structure]
wherein:
each X is independently C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
k is 0, 1, 2, 3, 4, or 5;
Z is CH or N;
L is —NR¹—(CR³R⁴)ₙ—NR²—, —(CR³R⁴)ₙ—NR²—, —O—(CR³R⁴)ₙ—NR²—, [chemical structure]
wherein each R¹ and R² is independently H, C₁₋₆ alkyl, C₂₋₆ alkenyl, or C₆₋₁₂ aralkyl;
each R³ and R⁴ is independently H, C₁₋₆ alkyl, C₂₋₆ alkenyl, C₆₋₁₂ aralkyl, C₁₋₆ alkoxyl, C(═O)—C₁₋₆ alkyl, C₁₋₆ haloalkyl, hydroxyl, halo, nitro or cyano; or CR³R⁴ is C═O;
n is 1, 2, 3, or 4;
Y is [chemical structure]
wherein R⁵ is H or C₁₋₆ alkyl.

4. The compound of claim 1, wherein the compound is a compound of Formula V or a pharmaceutically acceptable salt, ester, prodrug or derivative thereof: [chemical structure]
wherein:
X is C₁₋₆ alkyl, C₁₋₆ alkoxyl, C(═O)—C₁₋₆ alkyl, C₁₋₆ haloalkyl, C₃₋₆ cycloalkyl, hydroxyl, halo, nitro or cyano;
Z is CH or N;
Y is [chemical structure]
wherein R⁵ is H or C₁₋₆ alkyl.

6. The compound of claim 1, wherein the compound is selected from the group consisting of: [chemical structure] [chemical structure] [chemical structure]
and pharmaceutically acceptable salts, esters, prodrugs and derivatives thereof.

7. The compound of claim 1, wherein the compound is selected from the group consisting of: [chemical structure]
and pharmaceutically acceptable salts, esters, prodrugs and derivatives thereof.

8. A pharmaceutical composition comprising a compound of claim 1 in a pharmaceutically acceptable carrier.

9. A method of treatment or prophylaxis of neurologic disorders comprising administering to a host in need thereof an effective amount of a compound of claim 1, optionally in a pharmaceutically acceptable carrier.

10. The method of claim 9, wherein the disorder is a neurodegenerative disorder.

11. The method of claim 9, wherein the neurologic disorder is neuropathic pain, stroke, traumatic brain injury, epilepsy, or other neurologic events.

12. The method of claim 9, wherein the neurologic disorder is associated with an increase or decrease in NMDA receptor activity.

13. The method of claim 9, wherein the neurologic disorder is a neuropsychiatric disorder.

14. The method of claim 9, wherein the neurologic disorder is a traumatic brain injury.

15. The method of claim 9, wherein the neurologic disorder is a concussion.

16. The method of claim 9, wherein the neurologic disorder is a blast injury.

17. The method of claim 9, wherein the compound is administered in combination with a pharmaceutically acceptable carrier.

18. The method of claim 9, wherein the compound is administered in combination or alternation with a second active agent.
